(19) United States
(12) Patent Application Publication
(10) Pub. No.: US 2009/0012515 A1
(43) Pub. Date: Jan. 8, 2009

(54) DEVICES, SYSTEMS AND METHODS FOR TREATING TISSUES

(76) Inventors: Peter A. Hoenig, Sudbury, MA (US); B. Stuart Trembly, Hanover, NH (US); Kenneth M. Jones, Wellesley Hills, MA (US); Laura L. Deming, Lunenberg, MA (US); Michael A. Ouradnik, Wayland, MA (US); Anthony R. Tremaglio, Jr., Waban, MA (US); Bryan R. Hotaling, Harvard, MA (US); James R. Varney, Maynard, MA (US)

Correspondence Address: LOWRIE, LANDO & ANASTASI, LLP, ONE MAIN STREET, SUITE 1100, CAMBRIDGE, MA, 02142, US

(21) Appl. No.: 11/774,367

(22) Filed: Jul. 6, 2007

(51) Int. Cl. A61B 18/18

(52) U.S. Cl. 606/33; 604/20

(57) ABSTRACT

Devices, systems and methods for treating a tissue infected with an organism are provided. In certain examples, the device includes an applicator energetically coupled to an electromagnetic energy source to provide electromagnetic energy to the tissue for treatment. The device may also include a controller electrically coupled to the electromagnetic energy source to implement one or more treatment methods.

### FIELD OF THE TECHNOLOGY

Embodiments of the technology disclosed herein relate generally to devices, systems and methods for treating tissues. More particularly, certain embodiments disclosed herein relate to devices, systems and methods for treating tissues infected with one or more organisms.

### BACKGROUND

Infectious diseases of tissues such as, for example, keratinized tissues are a difficult problem for medical treatment. Keratins are a class of scleroprotein that serve as the major protein components of hair, wool, nails, the organic matrix of the enamel of teeth, horns, hoofs, and the quills of feathers. These proteins generally contain large quantities of the sulfur-containing amino acids, particularly cysteine. Keratins provide a tough, fibrous matrix for the tissues in which they are found. These proteins are characterized as being extremely water insoluble. Because keratins contain few polar amino acids, there is little or no moisture content in the tissues they form. This presents difficulties for the medical treatment of infected keratinized tissues because medicaments are not easily delivered into this type of tissue.

There remains a need for better devices and methods to treat tissues infected with a pathogen.

### SUMMARY

In accordance with a first aspect, a system constructed and arranged to treat a mammalian tissue infected with an organism is provided. In certain examples, the system comprises an electromagnetic energy source, an applicator operatively coupled to the electromagnetic energy source and configured to deliver electromagnetic energy to the mammalian tissue, and a controller operatively coupled to the electromagnetic energy source and configured to determine a treatment dose of the mammalian tissue and to provide for delivery of the determined treatment dose of the electromagnetic energy to the mammalian tissue.

In certain examples, the system may further comprise a temperature sensor operatively coupled to the controller and configured to detect a treatment temperature. In other examples, the applicator may comprise an adaptor constructed and arranged to be placed in proximity to the tissue to deliver the electromagnetic energy. In some examples, the adaptor may be constructed and arranged to conform to a digit surface. In yet other examples, the digit surface may be a nail or nail bed. In certain examples, the applicator comprises a tissue interface configured to receive a bolus. In other examples, the tissue interface may be configured to provide impedance matching of the mammalian tissue and the applicator. In some examples, the applicator comprises a flexible substrate configured for a single use. In certain examples, the controller may be configured to provide pulses of the determined treatment dose. In some examples, the controller may be configured to provide the determined treatment dose to provide continuous heating of the tissue until the mammalian tissue reaches a treatment temperature. In other examples, the controller may be configured to halt delivery of the determined treatment dose once the mammalian tissue reaches the treatment temperature. In certain examples, the controller may be configured to continue delivery of the determined treatment dose once the mammalian tissue drops below the treatment temperature. In some examples, the controller may be configured to deliver the determined treatment dose for a selected time. In certain examples, the adaptor may be constructed and arranged to smooth the distribution of energy. In other examples, the adaptor may be constructed and arranged to treat at least two nails simultaneously.

In accordance with another aspect, a method of treating a tissue of a mammal infected with an organism is disclosed. In certain examples, the method comprises a first step comprising determining a treatment dose of electromagnetic energy that a mammal can tolerate, and a second step comprising exposing the tissue to the determined treatment dose for a treatment time.

In some examples, the method may further comprise a third step comprising halting exposure of the tissue to the determined treatment dose once the tissue reaches a first temperature. In other examples, the method may further comprise a fourth step comprising continuing exposure of the tissue to the determined treatment dose once tissue temperature drops below the first temperature. In some examples, the steps of halting and continuing are repeated for the treatment time. In certain examples, the method may further comprise obtaining a culture of an organism infecting the tissue to assess efficacy of treatment. In some examples, the method may comprise assessing efficacy of treatment in less than one month or two weeks following the treatment. In some examples, the method may comprise exposing the tissue to one or more power levels of electromagnetic energy to determine the rate of heating to the first temperature.

In certain examples, the method may comprise removing an onycholytic portion of a nail before treatment. In some examples, the method may further comprise placing a biocompatible material over treated tissue to block access of infectious agents after treatment. In other examples, the biocompatible material may be toxic to infectious agents. In certain examples, the method may comprise delivering a drug to the infected tissue with the electromagnetic energy provided to the tissue by iontophoresis. In other examples, the method may further comprise delivering a drug to the infected tissue with the electromagnetic energy provided to the tissue by dielectrophoresis. In certain examples, the method may comprise exposing the tissue to the determined treatment dose for the treatment time from about five minutes to about thirty minutes. In some examples, the method may comprise increasing the first temperature during treatment based on a new tolerance level of the mammal. In other examples, the method may further comprise increasing temperature of the first temperature by inducing reactive hyperemia in the tissue. In certain examples, the method may further comprise increasing temperature of the first temperature by exposing the tissue to a coolant blown or sprayed on or encompassing the tissue. In some examples, the method may further comprise increasing temperature of the first temperature by exposing the tissue to a vibrating motion. In other examples, the determined treatment dose may also be effective to increase a nail growth rate.

In accordance with an additional aspect, a kit for treating an infected tissue is provided. In certain examples, the kit comprises an adaptor constructed and arranged to be coupled to an electromagnetic energy source and to deliver electromagnetic energy to an infected tissue. In some examples, the kit may also comprise a bolus configured to focus the electromagnetic energy to the infected tissue. In other examples, the kit may further comprise instructions for using the adaptor and the bolus to treat the infected tissue.

In certain examples, the adaptor may further comprise a tissue interface configured to receive the bolus. In some examples, the adaptor may be constructed and arranged to treat a nail. In other examples, the adaptor may be constructed and arranged to treat a hoof. In certain examples, the bolus may be configured to provide impedance matching of the infected tissue and the adaptor.

In accordance with another aspect, a system constructed and arranged to treat a digit surface tissue infected with an organism is disclosed. In certain examples, the system comprises an electromagnetic energy source, an applicator operatively coupled to the electromagnetic energy source and configured to deliver electromagnetic energy to the digit surface. In some examples, the applicator may comprise a tissue interface configured to receive a bolus, and an adaptor coupled to the applicator and constructed and arranged to conform to the digit surface. In other examples, the system may also comprise a controller operatively coupled to the electromagnetic energy source and configured to provide for delivery of a determined treatment dose of the electromagnetic energy to the digit surface.

In certain examples, the adaptor may be constructed and arranged to conform to a nail. In some examples, the adaptor may be constructed and arranged to conform to a hoof. In other examples, the tissue interface may be configured in combination with the bolus to smooth the distribution of the electromagnetic energy provided to the digit surface. In certain examples, the system may further comprise a temperature sensor operatively coupled to the digit surface and configured to detect a treatment temperature.

In accordance with an additional aspect, a system for treating a mammalian nail or hoof infected with an organism is disclosed. In certain examples, the system comprises an applicator constructed and arranged to deliver electromagnetic energy to a nail or a hoof, and a housing sized and arranged to receive a hand, a foot or a hoof of a mammal. In some examples, the housing comprises an electromagnetic energy source operatively coupled to the applicator, and a controller operatively coupled to the electromagnetic energy source and configured to determine a treatment dose of the nail or hoof and configured to provide for delivery of the determined treatment dose of electromagnetic energy to the nail or the hoof.

In certain examples, the applicator may comprise a plurality of adaptors to treat at least two adjacent digit surfaces on the hand, foot or hoof. In some examples, at least one adaptor of the plurality of adaptors may comprise a tissue interface configured to receive a bolus. In other examples, the tissue interface in combination with the bolus may be configured to provide impedance matching of the mammalian tissue and the applicator.

Additional aspects and features of the technology, and uses of such additional aspects and features, are disclosed in more detail herein.

### BRIEF DESCRIPTION OF THE FIGURES

Certain examples are described in detail below with reference to the accompanying figures in which:

FIG. 1 is block diagram of a device for treating tissue, in accordance with certain examples;

FIG. 2 is a schematic of an applicator including an adaptor, in accordance with certain examples;

FIG. 3 is a schematic of a device for treating tissue, in accordance with certain examples;

FIG. 4 is an example of a spacer in contact with a tissue, in accordance with certain examples;

FIG. 5 is an example of an adaptor including a container for a bolus, in accordance with certain examples;

FIG. 6 shows two energy profile graphs of an applicator without a bolus (top panel) and with a bolus (bottom panel), in accordance with certain examples

FIG. 7 is an example of a system for treating a tissue, in accordance with certain examples;

FIG. 8 is an example of a computer system suitable for use with the devices, systems and methods disclosed herein, in accordance with certain examples;

FIG. 9 is an example of a storage system, in accordance with certain examples;

FIG. 10 is a flow-chart of a protocol for treating a tissue, in accordance with certain examples;

FIG. 11 is a flow-chart of a protocol for treating a tissue, in accordance with certain examples;

FIG. 12 is an energy versus time graph showing treatment times and delay times, in accordance with certain examples;

FIG. 13 is a flow chart showing a calibration protocol, in accordance with certain examples;

FIGS. 14A and 14B show a housing enclosing a system suitable for delivering electromagnetic energy to a foot, in accordance with certain examples;

FIG. 15 shows a patient seated on a table with a foot resting on the housing shown in FIGS. 14A and 14B, in accordance with certain examples;

FIG. 16 is a block diagram of a device for treating an infected nail, in accordance with certain examples;

FIG. 17 is a block diagram of an applicator energetically coupled to two electromagnetic energy sources, in accordance with certain examples;

FIG. 18 is an insert configured to receive a tissue, in accordance with certain examples;

FIG. 19 is an insert configured to receive a tissue and disposed on a platform, in accordance with certain examples;

FIGS. 20A and 20B show two embodiments of disposing one or more agents on a tissue, in accordance with certain examples;

FIGS. 21A-25B show embodiments of disposing an applicator on a tissue, in accordance with certain examples;

FIG. 26A is a schematic of an adaptor comprising a flex circuit, in accordance with certain examples.

FIGS. 26B and 27 show an adaptor in contact with a toe, in accordance with certain examples;

FIGS. 28A-28C show various embodiments of a single-use adaptor, in accordance with certain examples;

FIG. 29 shows an illustrative device for performing iontophoresis or electrokinetic delivery of a substance, in accordance with certain examples;

FIG. 30 shows a device configured for delivery of electromagnetic energy and for iontophoresis or electrokinetic delivery of a substance, in accordance with certain examples;

FIG. 31 is a flow chart of an illustrative calibration protocol, in accordance with certain examples;

FIG. 32 is a flow chart of an illustrative treatment protocol, in accordance with certain examples;

FIGS. 33A-33F are photographs showing the large toe nail at various intervals after treatment, in accordance with certain examples;

FIG. 34 is a temperature profile graph during treatment of a toe nail for a fungal infection, in accordance with certain examples;

FIG. 35 is another temperature profile graph during treatment of a toe nail for a fungal infection, in accordance with certain examples;

FIG. 36 is a flow chart of another illustrative calibration protocol, in accordance with certain examples; and

FIGS. 37 and 38 are flow charts of another illustrative treatment protocol, in accordance with certain examples.

It will be recognized by the person of ordinary skill in the art, given the benefit of this disclosure, that certain dimensions, element or features in the figures may have been enlarged, minimized, distorted, shown not to scale or otherwise shown in a non-conventional manner to provide a better understanding of the technology disclosed herein.

### DETAILED DESCRIPTION

Certain illustrative embodiments and examples are described in more detail below to illustrate further some of the many configurations and applications of the technology disclosed herein.

In certain examples, the apparatus disclosed herein may be configured to deliver electromagnetic energy to a tissue for treatment of a particular disease or disorder affecting the tissue. In some examples, embodiments of the devices, systems and methods disclosed herein may be used to treat diseased tissue using electromagnetic energy or radiation. Treatment provides for improvement of symptoms and/or appearance by deactivating or killing of the organism or organisms infecting the tissue. For example, the organism may be thermally deactivated by delivering electromagnetic energy to a target area, which can be adjacent to or near the organism or may include the organism. Tissue surrounding the organism itself may also absorb energy or radiation and transfer thermal energy to the organism to deactivate the organism, and/or the organism can absorb directly the energy or radiation. Deactivation of the organism can render it unable to grow, reproduce and/or replicate. Deactivation can result from thermal destruction of the organism, from denaturing or partially denaturing one or more molecules forming the organism, from initiating a photobiological or photochemical reaction that attacks the organism, and/or from inducing an immune response that attacks the organism. In some examples, the electromagnetic radiation may result in killing of the organism.

In accordance with certain examples, the devices, systems and methods disclosed herein may be used to provide a determined treatment dose of electromagnetic energy, a deactivating dose of electromagnetic energy or a kill dose of electromagnetic energy. A “dose,” as used herein is defined by a combination of the treatment time and average temperature that is maintained on a surface of the target tissue during the treatment. A “therapeutic dose” is defined as a dose required for killing or disabling the pathogen cells. As used herein, “determined treatment dose” of electromagnetic energy refers to the case where the treatment dose is based on certain factors including, for example, subjective inputs based on subject responses. The treatment dose may be variable from subject to subject and may generally be determined by incrementally increasing the electromagnetic energy level until the subject becomes uncomfortable. In the case of humans, the subject may verbally or physically express a sensation of pain or heat. In the case of non-human mammals, the subject may attempt to remove or withdraw the area being exposed from the electromagnetic energy source. It is believed that by providing a determined treatment dose of electromagnetic energy, treatment may be more efficacious and may take less time. Once the determined treatment dose is identified, such dose may be programmed into a controller to effectuate treatment using the devices, systems and methods disclosed herein.

In certain examples herein that use a treatment dose, treatment may be performed for a fixed time or a variable time based on temperature measurements. For example, the target tissue temperature may be monitored to determine the average treatment temperature. In certain instances, an average tissue temperature of about 45-55° C. at a treatment time of at least 2-3 minutes provides a therapeutic dose. After initiation of treatment, the tissue temperature will increase up to a patient's tolerance level (referred to in certain instanced herein as a threshold temperature), based on subjective user inputs taking into account a patient's pain threshold, or up to a default safety maximum temperature, e.g., 53° C., and the treatment will then be halted. Treatment may be reinitiated once the tissue temperature falls below a certain value or once a defined period has passed. As the treatment proceeds, generally the patient may acclimate to the threshold temperature and will be able to tolerate a greater (higher) temperature. In this case, the threshold temperature may be increased and effectively the patient can control the temperature to maintain the temperature along the boundary of their pain threshold. Such a treatment process can provide a very effective therapeutic dose to treat the tissue. The increase in temperature during acclimation may be accomplished by treatment for a longer period, increasing the intensity of the electromagnetic energy applied to the tissue, focusing the electromagnetic energy or other suitable methods.

In some examples discussed herein, treatment may be provided at a determined treatment dose until the subject becomes uncomfortable or until the tissue reaches a selected threshold temperature, referred to in some instances herein as a “treatment temperature.” Treatment may be discontinued to permit the tissue temperature to fall below the treatment temperature, and then may be re-initiated for second treatment period at the maximum dose until the tissue temperature again rises to the treatment temperature. This process may be repeated iteratively until a desired treatment time is reached.

In some examples, the treatment temperature may be variable during the treatment. For example, during the course of administering the determined treatment dose, the subject may be able to tolerate a higher temperature due to, for example, desensitization of the area, increased blood flow and the like. In this situation, the treatment temperature may be increased such that more effective treatment may be effectuated. In other examples, the subject may not be able to tolerate the treatment temperature as treatment progresses, and the treatment temperature may be reduced prior to continuing further treatment. The exact treatment time may vary depending on the selected type of electromagnetic energy, and illustrative treatment times are discussed herein.

As used herein, “deactivating dose” refers to the amount of electromagnetic energy that can deactivate 80-99%, more particularly 95-99% or more of the organisms present in an infected tissue. Deactivation results in the organism being unable to replicate or survive but does not instantly kill the organism. As used herein “kill dose” refers to the amount of electromagnetic energy that can kill at least about 95% of the organisms present in an infected tissue. In contrast to deactivation, killing of the organism is substantially instantaneous and may be caused by superheating and exploding of the organism, leakage of ions or water into the cell or rupture of the cell membrane and/or cell wall.

As used herein, the term “electromagnetic energy” is used broadly and is intended to include gamma rays (wavelength less than about 10⁻⁹ cm), X-rays (wavelength from about 10⁻⁷ cm to about 10⁻⁹ cm), ultraviolet light (wavelength of about 4×10⁻⁵ cm to about 10⁻⁷ cm), visible light (wavelength of about 7×10⁻⁵ cm to about 4×10⁻⁵ cm), infrared light (wavelength of about 0.01 cm to about 7×10⁻⁵ cm), microwave radiation (wavelength of about 10 cm to about 0.01 cm), radio waves (wavelength of greater than about 10 cm) and any wavelength or energy between these illustrative types of electromagnetic energy, e.g., sound waves in various forms or from devices such as ultrasound devices having a wavelength of about 1.5 mm. The exact form of the electromagnetic energy used to treat tissue may vary depending on numerous factors including the wavelength of the electromagnetic energy, the tissue to be treated, treatment times, dosage and the like. Illustrative forms and devices for providing electromagnetic energy to tissue for treatment are discussed herein, and additional suitable forms and devices for providing electromagnetic energy to tissue for treatment will be readily selected by the person of ordinary skill in the art, given the benefit of this disclosure.

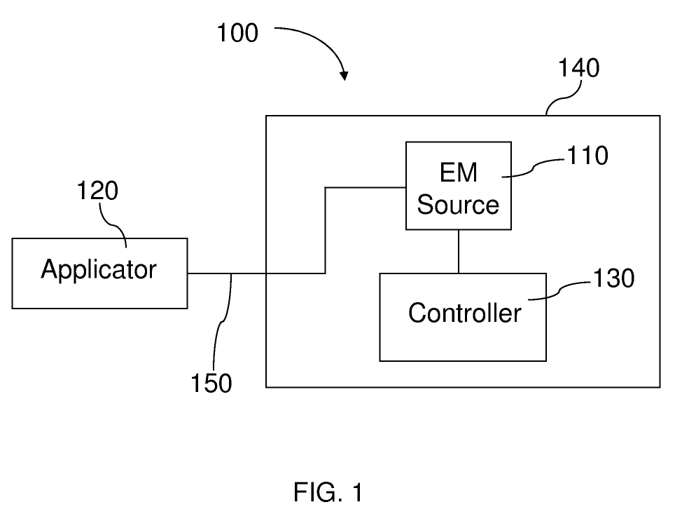

In accordance with certain examples, an illustrative apparatus for providing electromagnetic energy to a tissue is shown in FIG. 1. The apparatus 100 includes an electromagnetic energy source 110 energetically coupled to an applicator 120. As used herein “energetically coupled” refers to the configuration where energy generated or provided by the electromagnetic energy source 110 can be transmitted to the applicator 120 and on to a tissue. In certain embodiments, the apparatus 100 may also include a controller 130 that is electrically coupled to the electromagnetic energy source 110 and optionally to the applicator 120. In certain examples, the electromagnetic energy source 110 and the controller 130 may be in or on a housing 140. The applicator 120 is typically, though not necessarily, located external to the housing 140 and is energetically coupled to the electromagnetic energy source 110 through interconnect or cable 150. In certain examples, during operation of apparatus 100, the applicator 120 may be placed on or near the tissue to be treated and electromagnetic energy may be provided to the applicator 120 from the electromagnetic energy source 110 through the interconnect or cable 150.

In accordance with certain examples, the exact configuration of the applicator may vary depending on the type of electromagnetic energy to be delivered to the tissue. In examples where the applicator is configured to deliver gamma radiation or X-rays to the tissue, the applicator may be a tube or cable with suitable shielding to prevent unwanted gamma radiation from exiting the cable 150 while allowing gamma radiation or X-rays to exit at a terminus of the applicator. In examples where the applicator is configured to deliver UV light, visible light or infrared radiation to the tissue, the applicator may be a fiber optic device or a light pipe that allows for transmission of the UV or visible light from a light source to the tissue. In examples where the applicator is configured to deliver microwave radiation or radio waves to the tissue, the applicator may be a coaxial cable, waveguide or the like that permits passage of microwaves or radio waves from a source to the tissue. Other embodiments are discussed herein for applicator configurations that provide for delivery of different types of electromagnetic energy.

In accordance with certain examples, the applicator may be configured for delivery of electromagnetic energy to the skin to treat an infection of the skin or to prevent an infection of the skin, e.g., in or near a skin wound, in a human or non-human mammal such as, for example, a cow, sheep, or horse. In certain examples, the applicator may be configured to deliver electromagnetic energy to treat a bacterial skin infection such as, for example, cellulitis, erythrasma, folliculitis, skin abscesses, carbuncles, Hidradenitis suppurativa, impetigo, necrotizing skin infections or Staphylococcal scalded skin syndrome. In other examples, the applicator may be configured to deliver electromagnetic energy to treat a blistering disease such as, for example, bullous pemphigoid, dermatitis herpetiformis, or pemphigus. In yet other examples, the applicator may be configured to deliver electromagnetic energy to treat a fungal skin infection such as, for example, candidiasis, ringworm, tinea versicolor, tinea pedis or onychomycosis. In still additional examples, the applicator may be configured to deliver electromagnetic energy to treat an itching and noninfectious rash such as, for example, contact dermatitis, atopic dermatitis, seborrheic dermatitis, nummular dermatitis, generalized exfoliative dermatitis, stasis dermatitis, perioral dermatitis, pompholyx, a drug rash, erythema multiforme, erythema nodosum, granuloma annulare, itching, keratosis pilaris, lichen planus, pityriasis rosea, psoriasis, rosacea, Stevens-Johnson Syndrome, toxic epidermal necrolysis or other dermatalogical disorders such as, for example, dry nail. In certain examples, the applicator may be configured to deliver electromagnetic energy to treat parasitic skin infections such as, for example, creeping eruption, lice infestation, or scabies. In yet other examples, the applicator may be configured to deliver electromagnetic energy to treat a viral skin infection, such as molluscum contagiosum or warts. In other examples, the applicator may be configured to treat psoriatic nail disease following nummular dermatitis.

In certain examples, the treatment methods and devices disclosed herein may be used with one or more therapeutics or other compositions designed to prevent or reduce the likelihood of reinfection. Illustrative materials include antibiotics, antifungals, tissue sealants, tissue barriers and the like. It will be within the ability of the person of ordinary skill in the art, given the benefit of this disclosure, to select suitable compositions and devices to discourage or prevent reinfection of a tissue.

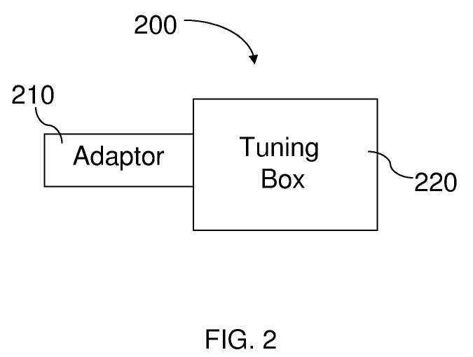

In accordance with certain examples, the applicator may include an adaptor that is sized and arranged to fit over the area of the tissue to be treated. For example and referring to FIG. 2, an applicator 200 may include an adaptor 210 that is energetically coupled to an electromagnetic energy source (not shown). The adaptor 210 is typically constructed and arranged to mirror the shape of the area of the tissue to be treated, e.g., if the area to be treated is circular, then the adaptor may be constructed, or trimmed, to mirror the circular shape. Suitable materials for use in adaptors include, but are not limited to, metals, metal alloys, ceramics, plastics, polymers, conductive polymers and the like. The adaptor may be placed over the area to be treated and electromagnetic energy may be provided to the area through the adaptor using one or more of the methods disclosed herein.

In certain examples, the adaptor may be disposable such that subsequent to treatment, the adaptor may be removed or disconnected from the applicator and discarded. The use of a disposable adaptor may provide significant benefits including, but not limited to, simple and cheap adaptors for single use, the lack of having to sterilize adaptors subsequent to use and the ability to use a new adaptor for each treatment and each subject to minimize any cross-contamination. The exact configuration of a disposable adaptor may vary depending on the nature and type of electromagnetic energy to be delivered and illustrative disposable adaptors are discussed in more detail herein.

In certain examples and referring to FIG. 2, the applicator 200 may also include a tuning box 220 that may be filled with a selected material such that the frequency of the electromagnetic energy provided to the adaptor may be further controlled. For example, the tuning box 220 may be filled with a gel or a sol material to tune further the frequency of the energy that passes through the applicator and/or through the adaptor. In some examples, a material may be added to the tuning box such that the impedance matching is accomplished at a particular frequency. While it is not required to configure the adaptor to be impedance matched, impedance matching may provide certain advantages, as discussed in more detail below. The exact material used in the tuning box can vary depending on the electromagnetic energy to be delivered. Illustrative materials for placement in the tuning box include a gel, such as, for example, commercially available lubricating jellies or a tissue-equivalent “phantom,” a fluid, e.g., water, acetone, methanol, ethanol, non-polar hydrocarbon based solvents, etc., or mixtures or combinations of any of the preceding substances, or a solid, e.g., a foam, fiber, glass, plastic or the like, Other suitable materials will be readily selected by the person of ordinary skill in the art, given the benefit of this disclosure.

In accordance with certain examples, the interconnect or cable 150 that provides for energetic coupling from the electromagnetic energy source to the adaptor may be positioned within tuning box 220 such that any electromagnetic energy passes through the tuning box on its way to the adaptor. In some examples, the energy passes through the tuning box but is not transferred from the tuning box to the adaptor, e.g., the cable runs through the center or some portion of the tuning box. In other examples, the energy is transferred to the tuning box, which passes the energy to the adaptor for delivery of the tissue to be treated.

In accordance with certain examples, the apparatus disclosed herein may also include or be configured to work with or receive a temperature sensor to monitor the temperature of the tissue to be treated. In certain examples, the exact configuration of the temperature sensor may vary depending on many factors including, but not limited to, the tissue to be treated, the type of electromagnetic energy to be used, the level of electromagnetic energy delivered, the configuration of the applicator or the like. In some examples, a temperature sensor such as those commercially available from Luxtron (Santa Clara, Calif.) may be used. In certain examples, the temperature sensor may be a thermocouple. In other examples, the temperature sensor may be a fiber optic thermometry sensor, a fluorescence based sensor or a radiation thermometry sensor. Additional suitable temperature sensors will be readily selected by the person of ordinary skill in the art, given the benefit of this disclosure.

In accordance with certain examples, the temperature sensor may be placed on the tissue to be treated to monitor the tissue temperature during treatment. Should the temperature of the tissue exceed a threshold value, i.e., a treatment temperature, the applicator may stop delivery of electromagnetic energy for a selected period. In certain examples, electromagnetic energy may be delivered until the temperature of the tissue reaches a desired temperature to provide for optimal treatment of the tissue. In other examples, delivery of the electromagnetic energy is constant or pulsed, but treatment is not halted prior to delivery of a selected dose unless the tissue temperature exceeds a threshold temperature.

In accordance with certain examples, once the tissue reaches a desired or threshold temperature, the tissue may be cooled either passively or actively. In configurations where passive cooling is used, the electromagnetic energy source may be switched off for a period to allow thermal transfer from the tissue to the surrounding environment. Alternatively, the electromagnetic energy source may stay on but the electromagnetic energy may be blocked from exiting the applicator and being delivered to the tissue. In embodiments where active cooling is used, heat may be removed from the tissue by placing a heat sink, fan, ice, ice pack or other device or material on the tissue to increase the temperature gradient between the tissue and the surrounding environment. In some examples, a device utilizing the Peltier effect may be employed to reduce the temperature of the tissue rapidly so that treatment may be continued and overall procedure time may be reduced. Additional methods and devices for lowering the temperature of a tissue to a desired value or below a threshold value will be readily selected by the person of ordinary skill in the art, given the benefit of this disclosure. Illustrative examples of active cooling, include, but are not limited to, contact conduction cooling, evaporative spray cooling, convective air flow cooling, a water jacket, a plate and pump configured to circulate a cooling fluid through the plate, a spray cooling device that uses cryogen, water, or air as a coolant, and combinations thereof.

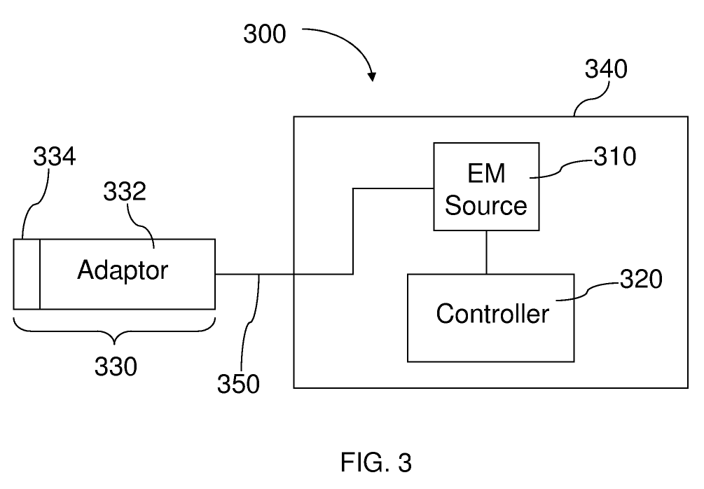

In certain examples, the temperature sensor may be electrically coupled to the controller such that treatment may be halted if the temperature of the tissue exceeds a threshold temperature or reaches a desired temperature. In other configurations, the temperature sensor may be an integral part of the applicator such that placement of the applicator on the tissue also results in bringing the temperature sensor into thermal communication with the tissue. An example of this configuration is shown in FIG. 3. The apparatus 300 includes an electromagnetic energy source 310, a controller 320 electrically coupled to the electromagnetic energy source 310, and an applicator 330 energetically coupled to electromagnetic energy source 310 through interconnect or lead 350. The apparatus 300 may also include a housing 340 which encloses the electromagnetic energy source 310 and the controller 320. The applicator 330 may include an adaptor 332 and a temperature sensor 334. In certain examples, the temperature sensor 334 may be detachable or removable from the adaptor 332 to facilitate cleaning of the applicator 330. Though temperature sensor 334 is shown on the terminus of the adaptor 332 in FIG. 3, the temperature sensor 334 may be positioned at any location on the adaptor 332 so long as the temperature sensor can detect the temperature of the tissue to be treated.

In certain examples, the temperature sensor is not in direct contact with the tissue to be treated, but is instead above, below or beside the tissue to be treated. In configurations where the temperature sensor is not in direct contact with the tissue, a lookup table or algorithm may be used to calculate or extrapolate the temperature of the tissue based on the detected temperature above, below or beside the tissue. For example, where the tissue to be treated has a small surface area, e.g., a small toenail or a small area of the skin or other organ, it may not be possible to place both the temperature sensor and the adaptor in contact with the tissue. The temperature sensor may be placed near the tissue, however, and the temperature properties or profile of a medium between the tissue and the temperature sensor may be used to extrapolate the temperature of the tissue. This medium may be a fluid, such as air, a liquid or a solution, may be a gel or sol, may be a foam or may be other suitable materials whose temperature properties are known or may be determined.

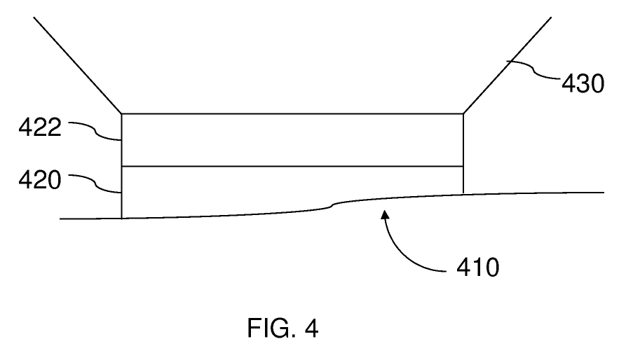

In accordance with certain examples, one or more spacers may be placed between the tissue and the adaptor and/or between the tissue and the temperature sensor. For example and referring to FIG. 4, a tissue surface 410 is in contact with spacers 420, 422, which are in contact with adaptor 430. In some embodiments, at least one surface of the spacer 420 may be conformable, compressible or expandable such that it can conform to the shape of the tissue surface to be treated. In the example shown in FIG. 4, the surface of spacer 420 that rests against the tissue surface 410 has conformed to the tissue surface 410. The use of a spacer may provide more uniform delivery of electromagnetic energy to the tissue when the surface or surfaces of the tissue are uneven. Suitable materials for use as a spacer includes, but is not limited to, metals, metal alloys, elastomers, plastics, polymers and the like. In some examples, one or more materials consisting of spacers separating volumes of air, e.g., a honeycomb material, where the spacers may be any of the illustrative materials listed above, may be used. In some configurations, the spacer may be selected so that it transmits energy from the adaptor to the tissue without altering the frequency or level of the energy. In other configurations, the spacer may be selected such that is alters the frequency or level of the energy prior to the energy being delivered to the tissue. It will be within the ability of the person of ordinary skill in the art, given the benefit of this disclosure, to use a spacer of a selected material with the apparatus disclosed herein.

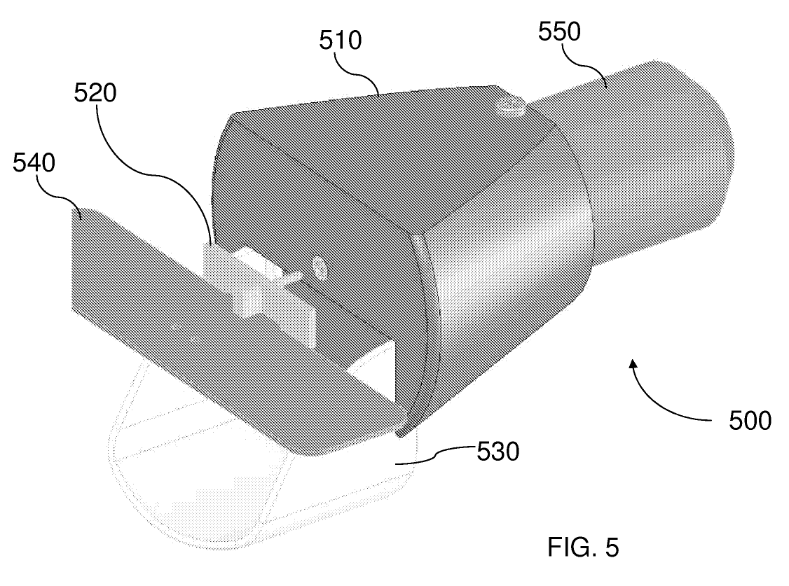

In accordance with certain examples, the applicator may be configured for delivery of electromagnetic energy to one or more nails of a human or non-human mammal. As discussed herein, treatment of keratinized tissue, such as that found in human nails or in the nails of non-human mammals such as sheep and horses, can be difficult. The devices, systems and methods disclosed herein may be used to provide electromagnetic energy to the nails and/or nail beds to improve the overall appearance of the nails. Such treatment may be performed, for example, to deactivate or kill pathogens infecting the nail and/or nail bed or to improve the overall appearance of the nail by preventing pathogens from infecting the nail or the nail bed. An illustrative example of an applicator that may be used to treat the nail is shown in FIG. 5. The applicator 500 comprises an adaptor 510 that includes a tuning box. The adaptor 510 is electrically coupled to an electromagnetic energy source (not shown) through cable 550. The applicator 500 also includes an end-cap 520 that may conform to the shape of the nail. The end-cap 520 may be electrically coupled to the adaptor 510. The applicator 500 also includes a tissue interface 530 configured to receive a bolus. The use of a bolus is discussed in more detail below. A nail may be positioned above the tissue interface 530 and in contact with the end-cap 520. An over-mold 540 may be placed on the top of the nail and may act to retain one or more temperature probes (not shown) placed on the nail. During operation of the applicator 500, electromagnetic energy may be delivered through cable 550 to end-cap 520 and into the nail for treatment of the nail tissue.

In accordance with certain examples and as discussed above, the electromagnetic energy delivered to the nail tissue may be any of the illustrative energy types discussed herein. It will be recognized by the person of the ordinary skill in the art, given the benefit of this disclosure, that the configuration of the end-cap 520 may vary depending on the type of electromagnetic energy to be delivered. The exact configuration of the end-cap is not critical so long as the end-cap can deliver a selected type of electromagnetic energy to the tissue to be treated. In embodiments where radio waves or microwaves are to be delivered, the end-cap may be configured as an antenna, wave guide, conductor or the like. In embodiments where a sound wave, e.g., a sound wave from an ultrasound device, is to be delivered, the end-cap may be configured with a sound transmitter. In embodiments where infrared, visible or ultraviolet light is to be delivered, the end-cap may be configured as a light-pipe, a fiber optic device, a light emitting diode, a laser diode, an incandescent source, a fluorescent source, an assembly of reflectors or other devices that may be used to deliver light. In embodiments where the X-rays or gamma rays are to be delivered, the end-cap may be configured as an opening in a lead-shielded cable, a guide, a cone, or a collimator, that is energetically coupled to an X-ray or gamma ray source. Other configurations for an end-cap to deliver a selected type of electromagnetic energy to a nail or other tissue will be readily selected by the person of ordinary skill in the art, given the benefit of this disclosure.

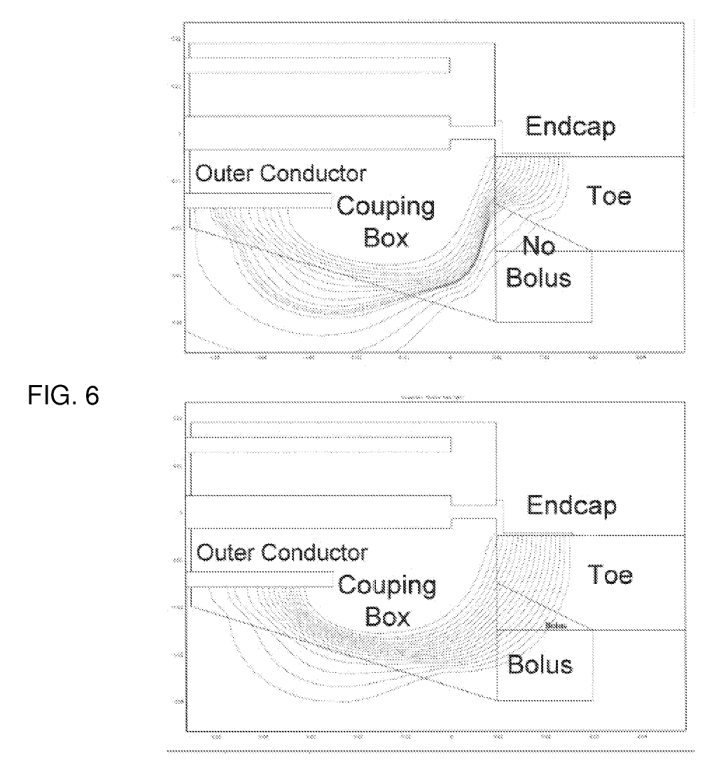

In certain examples, a bolus may be inserted into the tissue interface 530. By using a bolus underneath the tissue to be treated, e.g., a nail infected with a fungus, the electromagnetic energy delivered to the tissue may be more uniform. It is thought that the bolus provides tuning of the electromagnetic energy to provide a more uniform distribution of energy to a parallel path for electric field lines, thereby reducing their concentration at an undesired location within tissue; such a concentration of field lines may cause unwanted effects such as, for example, local overheating. For example and referring to FIG. 6, a simulation is shown with an applicator comprising an end-cap electrically coupled to a coaxial cable to provide microwave energy that may be used to treat a nail. This simulation was performed by solving Laplace's Equation for voltage, with a voltage difference enforced between the end-cap and the outer conductor of the coaxial cable. As can be seen in the top graph, in the configuration where no bolus is used, the electromagnetic energy delivered to the nail is non-uniform. This result may cause unwanted heating of the tip of the toe. By using a bolus, (bottom graph in FIG. 6) the energy that is delivered to the toe is more uniform. In addition, by using a bolus, the level of energy delivered to the applicator may be reduced due to the increased efficiency of delivery of the electromagnetic energy. For example, the level of microwave energy may be reduced by about 50% or more due to more uniform delivery of the energy, e.g., in the case of microwave energy, the energy provided to the applicator may be reduced from about 36 Watts to about 10 Watts without any substantial reduction in the amount of energy delivered to tissue. In certain cases, by using a bolus, the fraction of power reflected from the applicator may be reduced. For example, a measurement made with a dual directional coupler and two power meters showed that the fraction of microwave reflected power was reduced from 18% to 3% by using a bolus.

In accordance with certain examples, suitable materials for the bolus may vary depending on the type of electromagnetic energy to be delivered. In certain examples, the bolus has similar physical properties as those of the tissue to be treated, e.g., a similar water content, etc. Illustrative materials for use as a bolus include gelatin, collagen, agarose, a lubricating jelly, water, an ultrasound gel pad and similar materials. In certain examples, the bolus may be cast in a mold or die that has a similar size and geometry as that of the tissue interface 530. Alternatively, the bolus may be cut to shape from a larger bolus. In examples where a kit is employed, the bolus may be included in the kit and configured to be placed in the tissue interface without prior cutting or shaping by the operator. It will be within the ability of the person of ordinary skill in the art, given the benefit of this disclosure, to select, make and/or use a bolus with the devices, systems and methods disclosed herein.

In accordance with certain examples, the end-cap 520 may be configured to overlie and/or surround the tissue to be treated. In certain examples, the end-cap may be constructed or trimmed to be substantially the same shape as the tissue to be treated. In some examples, the end-cap may be electrically coupled to an interconnect or cable 550 so that electromagnetic energy may be transmitted from the cable 550 to the end-cap 520 and delivered to the nail tissue. The exact material used to construct the end-cap may vary depending on the type of electromagnetic energy to be delivered to the tissue. In examples where the electromagnetic energy to be delivered is radio waves or microwaves, the end-cap may be constructed from a conductive material, such as a metal, metal alloy, plastic or the like. In examples where the electromagnetic energy to be delivered to the tissue is infrared, visible or ultraviolet energy, the end-cap may include a fiber optic device to transmit the light. In examples where the electromagnetic energy to be delivered to the tissue is X-rays or gamma rays, the end-cap may include an opening for transmitting or focusing X-rays or gamma rays. Additional materials and configurations for an end-cap constructed and arranged to deliver a selected electromagnetic energy will be readily selected by the person of ordinary skill in the art, given the benefit of this disclosure.

In accordance with certain examples, the over-mold 540 may be used to retain one or more additional devices on the tissue and to facilitate proper placement of the applicator for treatment. In certain examples, the over-mold 540 may be used to hold a temperature sensor in place. In other examples, the over-mold 540 may be impregnated or coated with a therapeutic to provide additional treatment of the tissue. In some examples, the over-mold 540 may include a dye or agent that can facilitate transfer of the electromagnetic energy to the tissue. For example, a dye may be used to provide for increased absorption of energy from the applicator. The material or materials used in the over-mold may vary depending on the type of electromagnetic energy to be delivered, and, preferably, the materials do not substantially interfere with delivery of the electromagnetic energy to the tissue. In certain examples, the over-mold includes a material such as a silicone, a plastic or an elastomer, any of which may include an adhesive to retain the over-mold in position after placement on the tissue. Illustrative commercially available devices suitable for use as an over-mold include, but are not limited to, surgical tape, an adhesive bandage, a clear plastic film, a foil or the like. In other examples, the over-mold may be used to shield tissues that are not being treated from the electromagnetic energy. Such over-molds may be effective to absorb the electromagnetic energy or to otherwise prevent exposure of any underlying tissues to the electromagnetic energy.

In accordance with certain examples, when the applicator 500 shown in FIG. 5 is used, a bolus may be placed in container 530, a toe with an infected nail may be placed on top of the container 530, and the tip of the toe typically rests against the tuning box 510. A temperature sensor is placed on the nail tissue to be treated. The end-cap 520 may be brought into contact with the nail and over-mold 540 acts to hold the temperature sensor on the nail tissue. The exact methodology used to treat the nail depends on the electromagnetic energy to be delivered to the nail tissue, and illustrative methods are discussed in more detail herein. In certain examples, a controller may be operative to switch an electromagnetic energy source on, and energy may be delivered through end-cap 520 to the nail tissue to be treated.

In accordance with certain examples, the controller of the apparatus disclosed herein may be a simple device, such as a mechanical on/off switch. In other embodiments, the on/off switch may include a mechanical timer or timing circuit that automatically turns the apparatus off after a certain period from switching the apparatus on. For example, in configurations where the apparatus is designed for home use, depression of the on/off switch may cause transmission of electromagnetic energy through an applicator for a selected amount of time. The timer or timing circuit may be designed to automatically switch the electromagnetic energy source off after a selected period. In the alternative, the subject being treated with the electromagnetic energy may have control over treatment such that they can manually turn on and turn off the electromagnetic energy. In other configurations, the temperature sensor may be electrically coupled to the controller and once the tissue reaches a selected or threshold temperature, the controller may stop delivery of electromagnetic energy for a desired period.

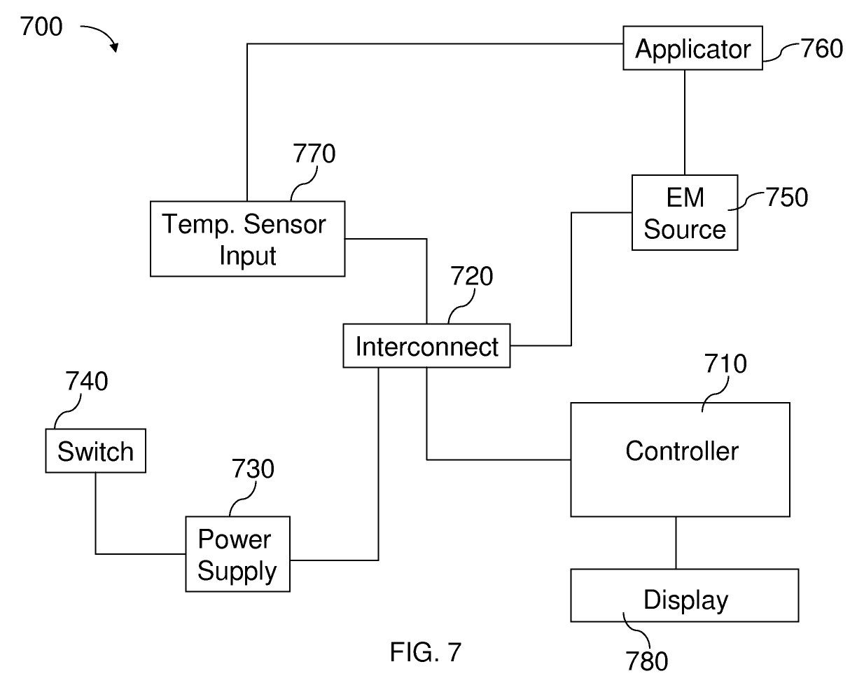

In other configurations, the controller may include a processor, associated circuitry and the like. An illustrative configuration for a controller in an apparatus is shown in FIG. 7. The controller 710 of the apparatus 700 is electrically coupled with the other components of the apparatus through an interface or interconnect 720, which typically is a bus such as a serial bus. The apparatus 700 also includes a power supply 730 electrically coupled to a switch 740. The apparatus 700 also includes an electromagnetic energy source 750 energetically coupled to an applicator 760. The applicator 760 may include or be used with a temperature sensor (not shown) which sends signals to temperature sensor input 770. In operation of apparatus 700, the controller 710 sends and receives signal from the various components of the apparatus. For example, the controller 710 may send a signal to initialize the electromagnetic energy source 750 to provide energy to the applicator 760. The temperature sensor input 770 can send signals to the controller 710 such that electromagnetic energy source 750 may be turned off if the temperature of the tissue exceeds a threshold temperature. The various parameters of the system, e.g., energy level, tissue temperature, etc., may be displayed on display 780 such that an operator may monitor treatment.

In accordance with certain examples, the controller 710 may include at least one processor optionally electrically coupled with one or more memory units. In certain examples, the controller may be a larger part of a computer system. The computer system may be, for example, a general-purpose computer such as those based on Unix, Intel PENTIUM-type processor, Motorola PowerPC, Sun UltraSPARC, Hewlett-Packard PA-RISC processors, or any other type of processor. In some examples, the processor may be an inexpensive processor that may be programmable to receive inputs and determine a treatment dose based on the received inputs. It should be appreciated that one or more of any type computer system may be used according to various embodiments of the technology. Further, the system may be located on a single computer or may be distributed among a plurality of computers attached by a communications network. A general-purpose computer system may be configured, for example, to perform any of the described functions including but not limited to: applicator control, temperature monitoring, data display and the like. It should be appreciated that the system may perform other functions, including network communication, and the technology is not limited to having any particular function or set of functions.

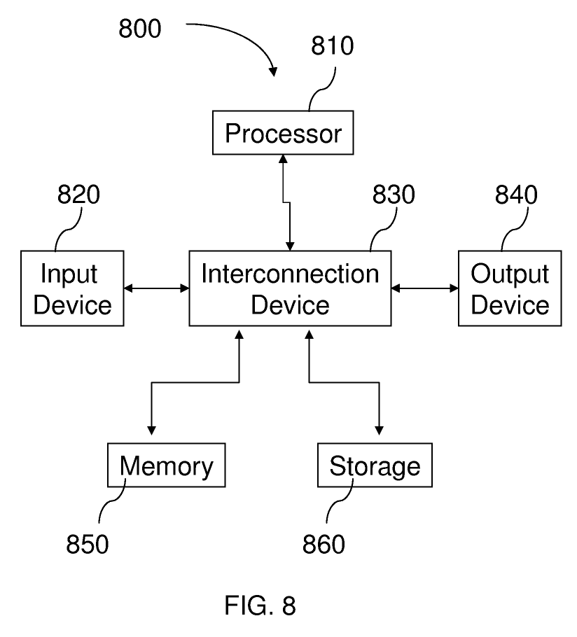

For example, various aspects may be implemented as specialized software executing in a general-purpose computer system 800 such as that shown in FIG. 8. The computer system 800 may include a processor 810 connected to one or more memory devices 850, such as a disk drive, memory, or other device for storing data. Memory 850 is typically used for storing programs and data during operation of the computer system 800. Components of computer system 800 may be coupled by an interconnection device 830, which may include one or more buses (e.g., between components that are integrated within a same machine) and/or a network (e.g., between components that reside on separate discrete machines). The interconnection device 830 provides for communications (e.g., signals, data, instructions) to be exchanged between system components of system 800. The computer system 800 typically is electrically coupled to the applicator (not shown) such that electrical signals may be provided from the applicator to the computer system 800 for storage and/or processing.

Computer system 800 may also include one or more input devices 820, for example, a keyboard, mouse, trackball, microphone, touch screen, manual switch (e.g., override switch) and one or more output devices 840, for example, a printing device, display screen, speaker. In addition, computer system 800 may contain one or more interfaces (not shown) that connect computer system 800 to a communication network (in addition or as an alternative to the interconnection device 830.

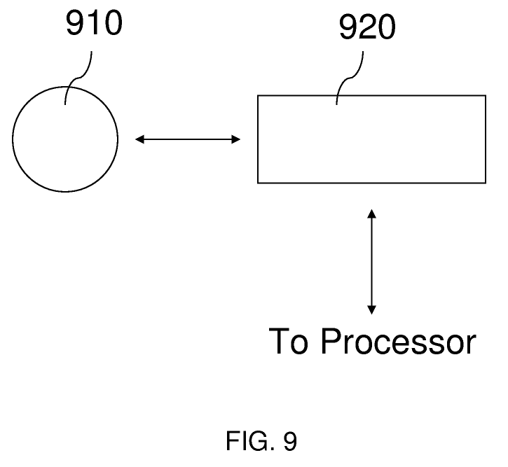

The storage system 860, shown in greater detail in FIG. 9, typically includes a computer readable and writeable nonvolatile recording medium 910 in which signals are stored that define a program to be executed by the processor or information stored on or in the medium 910 to be processed by the program. For example, the treatment dosing times, calibration methods, maximum dosages for a particular subject and the like used in certain embodiments disclosed herein may be stored on the medium 910. The medium may, for example, be a disk or flash memory. Typically, in operation, the processor causes data to be read from the nonvolatile recording medium 910 into another memory 920 that allows for faster access to the information by the processor than does the medium 910. This memory 920 is typically a volatile, random access memory such as a dynamic random access memory (DRAM) or static memory (SRAM). It may be located in storage system 860, as shown, or in memory system 850. The processor 810 generally manipulates the data within the integrated circuit memory 850, 920 and then copies the data to the medium 910 after processing is completed. A variety of mechanisms are known for managing data movement between the medium 910 and the integrated circuit memory element 850, 920, and the technology is not limited thereto. The technology is also not limited to a particular memory system 850 or storage system 860.

In certain examples, the computer system may also include specially-programmed, special-purpose hardware, for example, an application-specific integrated circuit (ASIC). Aspects of the technology may be implemented in software, hardware or firmware, or any combination thereof. Further, such methods, acts, systems, system elements and components thereof may be implemented as part of the computer system described above or as an independent component.

Although computer system 800 is shown by way of example as one type of computer system upon which various aspects of the technology may be practiced, it should be appreciated that aspects are not limited to being implemented on the computer system as shown in FIG. 8. Various aspects may be practiced on one or more computers having a different architecture or components than that shown in FIG. 8. Computer system 800 may be a general-purpose computer system that is programmable using a high-level computer programming language. Computer system 800 may be also implemented using specially programmed, special purpose hardware. In computer system 800, processor 810 is typically a commercially available processor such as the well-known Pentium class processor available from the Intel Corporation. Many other processors are available. Such a processor usually executes an operating system which may be, for example, the Windows 95, Windows 98, Windows NT, Windows 2000 (Windows ME), Windows XP or Windows Vista operating systems available from the Microsoft Corporation, MAC OS System X operating system available from Apple Computer, the Solaris operating system available from Sun Microsystems, or UNIX or Linux operating systems available from various sources. Many other operating systems may be used, and in certain embodiments a simple set of commands or instructions may function as the operating system.

In accordance with certain examples, the processor and operating system may together define a computer platform for which application programs in high-level programming languages may be written. It should be understood that the technology is not limited to a particular computer system platform, processor, operating system, or network. Also, it should be apparent to those skilled in the art, given the benefit of this disclosure, that the present technology is not limited to a specific programming language or computer system. Further, it should be appreciated that other appropriate programming languages and other appropriate computer systems could also be used.

In certain examples, the hardware or software is configured to implement cognitive architecture, neural networks or other suitable implementations. For example, a tissue database may be linked to the system to provide access to temperature tolerances for different tissues. Such configuration provides for use of the applicator with many different types of tissues, which may increase the flexibility and function of the devices, systems and methods disclosed herein.

One or more portions of the computer system may be distributed across one or more computer systems coupled to a communications network. These computer systems also may be general-purpose computer systems. For example, various aspects may be distributed among one or more computer systems configured to provide a service (e.g., servers) to one or more client computers, or to perform an overall task as part of a distributed system. For example, various aspects may be performed on a client-server or multi-tier system that includes components distributed among one or more server systems that perform various functions according to various embodiments. These components may be executable, intermediate (e.g., IL) or interpreted (e.g., Java) code which communicate over a communication network (e.g., the Internet) using a communication protocol (e.g., TCP/IP). It should also be appreciated that the technology is not limited to executing on any particular system or group of systems. Also, it should be appreciated that the technology is not limited to any particular distributed architecture, network, or communication protocol.

In accordance with certain examples, various embodiments may be programmed using an object-oriented programming language, such as SmallTalk, Basic, Java, C++, Ada, or C# (C-Sharp). Other object-oriented programming languages may also be used. Alternatively, functional, scripting, and/or logical programming languages may be used. Various configurations may be implemented in a non-programmed environment (e.g., documents created in HTML, XML or other format that, when viewed in a window of a browser program, render aspects of a graphical-user interface (GUI) or perform other functions). Certain configurations may be implemented as programmed or non-programmed elements, or any combination thereof.

In certain examples, a user interface may be provided such that a user may enter or recall a type of tissue, patient statistics, tissue condition or other data desired. For example, in instances where a patient has already received treatment, relevant treatment parameters may be recalled and reused without the need to determine maximum dosages or the like. Other features for inclusion in a user interface will be readily selected by the person of ordinary skill in the art, given the benefit of this disclosure.

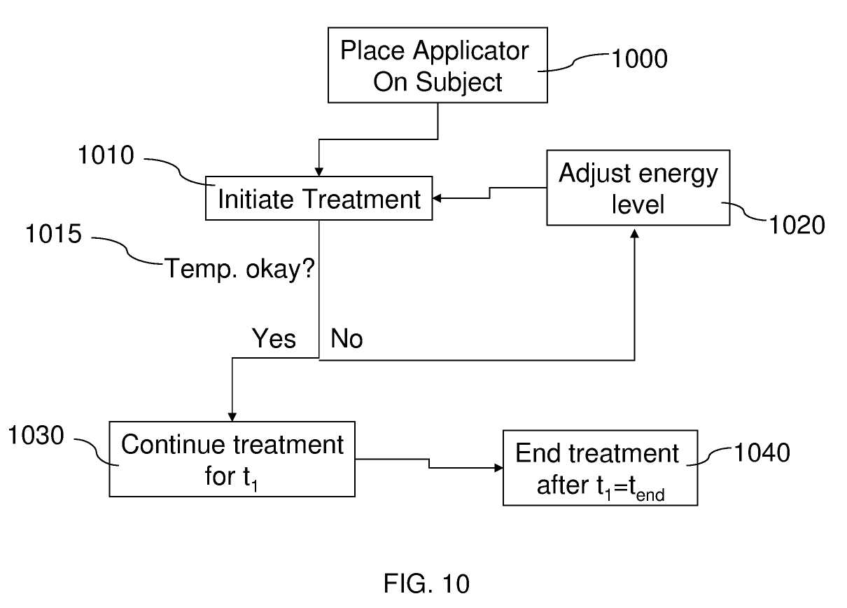

In accordance with certain examples, numerous methods may be implemented by the controller to deliver the electromagnetic energy to the tissue. In certain examples, the method may involve patient or subject input. For example, and referring to FIG. 10, the applicator may be placed on a subject 1000, and treatment may be initiated 1010. Using subjective or objective factors, such as subject feedback, it can be assessed whether or not the temperature is okay 1015. If the temperature is okay and the patient is comfortable, then treatment may continue for a time period t₁ until the treatment period t₁ is the same as a desired treatment interval t_{end}. If the dosage is too high such that the subject is uncomfortable, the energy may be reduced to a lower level 1020 and treatment may be re-initiated 1010 and continued for a time period t₁ until the treatment period t₁ is the same as a desired treatment interval t_{end}. It should be understood that the treatment period may include application of electromagnetic energy in a continuous or pulsed manner, as discussed in more detail herein.

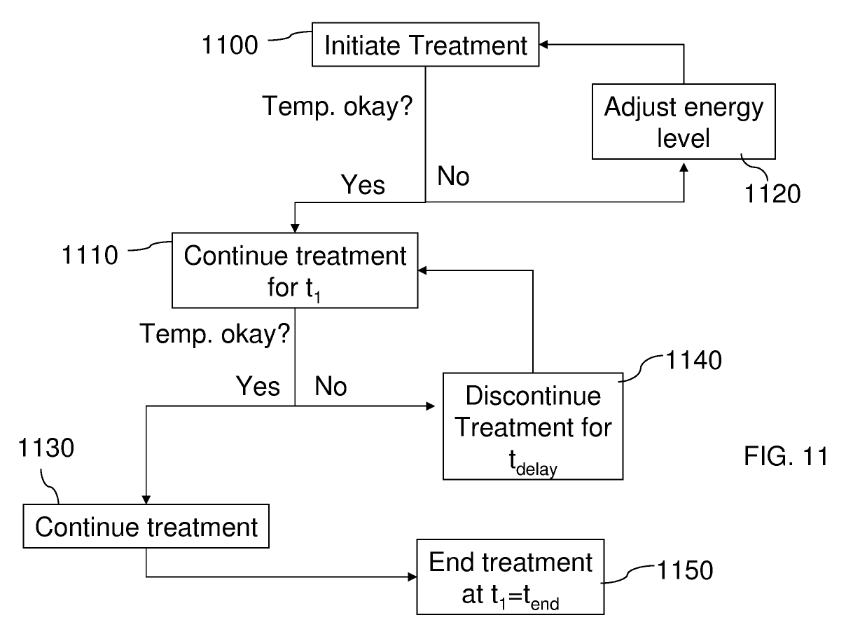

In certain examples, the temperature of the tissue may be monitored during application of the electromagnetic energy, as the temperature of the tissue may increase during treatment. An example of a method that implements this type of feedback is shown in FIG. 11. Treatment may be initiated 1100, and if the temperature of the tissue is suitable during treatment, then treatment may continue 1110 for treatment time t₁. If the temperature should exceed a threshold value or is uncomfortable to the patient, then the energy level can be adjusted 1120 to a lower level and treatment may be reinitiated. As treatment progresses, the tissue may heat up. If this situation occurs, then treatment may be discontinued 1140 for a delay time t_{delay} and can then be continued 1130 once the temperature of the tissue decreases to a suitable value. Should the temperature be within an acceptable value and should the total treatment time t₁ equal t_{end}, then treatment may be discontinued 1150.

In accordance with certain examples, treatment may be administered in a continuous manner by providing electromagnetic energy to the tissue for a selected period. For example, the controller may provide electromagnetic energy to the applicator for a continuous period to effectuate treatment of the tissue. Continuous treatment may be desirable where the tissue does not heat beyond a threshold temperature and where it is desirable to minimize total treatment time.

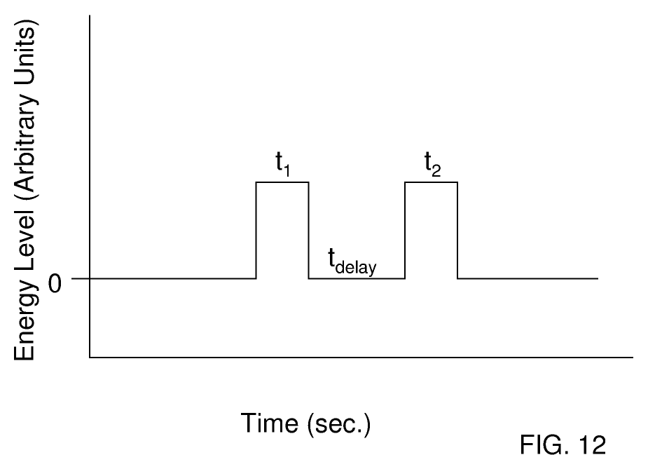

In accordance with certain examples, the treatment may be administered in a pulsed manner by using on/off cycles of continuously delivered energy. For example and referring to FIG. 12, a first pulse may be delivered by providing the energy for a time t₁. A delay period of t_{delay} occurs, which allows the temperature of the tissue to decrease. Following the delay period, another pulse of energy for a treatment time of t₂ may be delivered. This process of pulsing and delaying may be repeated for a sufficient time to provide treatment to the tissue. By controlling the duration of t₁, t₂, and t_{delay}, energy may be delivered to the tissue in a pulsed manner. In certain examples, the exact times for t₁, t_{delay} and t₂ may vary. In some examples, t₁, t_{delay} and t₂ are substantially the same. In other examples, t₁ and t₂ are substantially the same and t_{delay} may be greater than t₁ and t₂ to allow for tissue cooling. In other examples, t₁ may be greater than t₂ as t_{delay} may not be long enough to permit the temperature of the tissue to return all the way to its normal, resting temperature. In certain examples t₁ and t₂ are each about 10 seconds to about 20 seconds to about and t_{delay} is about 10 seconds to about 20 seconds.

In accordance with certain examples, the sum of the treatment times may be totaled such that treatment time continues until the total treatment time sums to a value t_{end}. The t_{end} value provides for approximately the same amount of treatment of each subject even if the t₁, t₂, t_{delay}, etc., times differ for different subjects. The total treatment time may vary depending on the exact type of electromagnetic energy delivered to the tissue. In certain examples, the total treatment time is no more than about 5 minutes. In other examples, however, the total treatment time may be about 5 minutes or greater. While in certain examples the total treatment time may be five minutes or less, the total time for a procedure involving administration of treatment to a patient may be substantially longer as the sum of the t_{delay} times may be a substantial value. In some examples, the total time for administering a single treatment to an individual varies from about 10 minutes to about 120 minutes, more particularly from about 20 minutes to about 90 minutes, e.g., about 30 minutes to about 60 minutes. The person of ordinary skill in the art, given the benefit of this disclosure, will be able to select suitable treatment and delay times for providing treatment of a particular tissue using a particular type of electromagnetic energy.

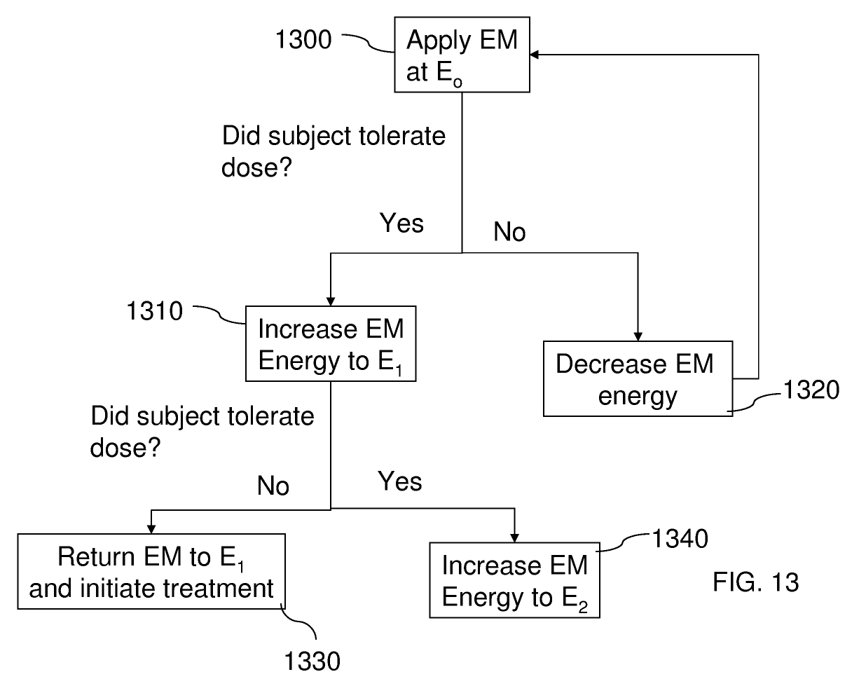

In accordance with certain examples, prior to treatment, one or more calibration steps may be performed to determine a maximum dose of the electromagnetic energy that a subject can tolerate. Without wishing to be bound by any particular scientific theory or this example, it is believed that heating of the tissue to a higher temperature using the methods and devices disclosed herein provides for more effective treatment. Such tissue heating is permitted to a level that still remains safe, e.g., up to about 57° C., so that the tissue cells are not killed or permanently damaged. With respect to treatment of fungus without damage to surrounding tissue, the tissue may be heated to a range between 43-57° C. and more preferably between 47-53° C. An illustrative calibration method is shown in FIG. 13. Electromagnetic energy may be applied 1300 at an initial energy Eₒ. If the patient or subject can tolerate the Eₒ energy level then the energy level may be increased 1310 to E₁. In the alternative, the power level may be adjusted or set such that the temperature changes (dT/dt) by a selected amount over a selected period. This dT/dt value is substantially linearly proportional to input power, which allows interpolation to select a desired dT/dt range by adjusting the power level. This process, for example, may be performed over a 10 second interval such that tissue heating to a tolerance level is not a factor. The power level that provides the desired dT/dt is the power level used for the balance of the treatment. Once that power level is determined, the first increase to the treatment temperature provides the tolerance of the patient, and from there it is possible to control the temperature limit based on their feedback. If the subject or patient is uncomfortable at the energy level E₁, then the energy may be returned 1320 to energy level Eₒ, or to an energy level between Eₒ an E₁, and treatment may be initiated. If the subject or patient is comfortable at the energy level E₁, then the energy level may be increased 1340 to E₂, and this process may be repeated to determine the maximum dosage that the subject or patient can tolerate. If the subject or patient was subjectively uncomfortable at energy level E₂, then the initial energy level may be reduced 1330 to Energy level E₁ or an energy level between E₂ an E₁ and optionally the step 1340 may be repeated to determine the maximum energy level that the subject or patient can tolerate. It is also to be appreciated that this methodology can be repeated, if the subject can tolerate the energy level E₂, to determine the energy level the subject can tolerate. It will be appreciated by the person of ordinary skill in the art, given the benefit of this disclosure, that the maximum dose that a subject can tolerate may change over the course of a single treatment and may change over the course of multiple treatments. In certain instances, a calibration step may be performed prior to each treatment, whereas in other instances a calibration step may be performed every other treatment (or other selected interval) to assess whether or not the maximum energy dose tolerated by a subject has changed. In addition, the treatment temperature may change during the course of treatment and may be adjusted on a subject by subject basis.

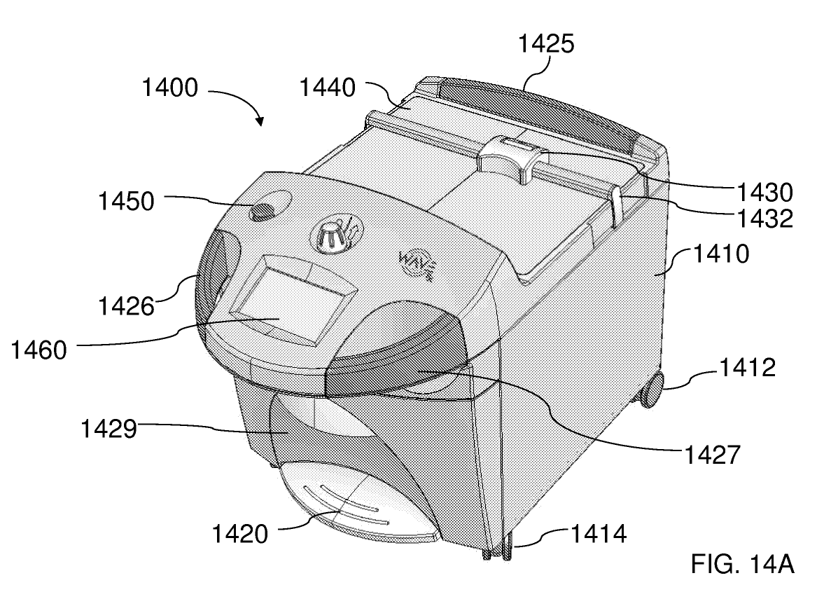

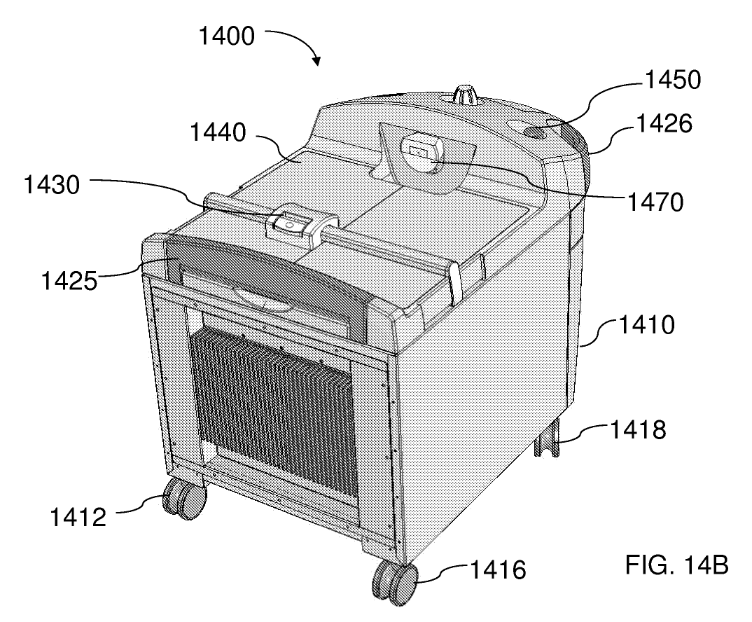

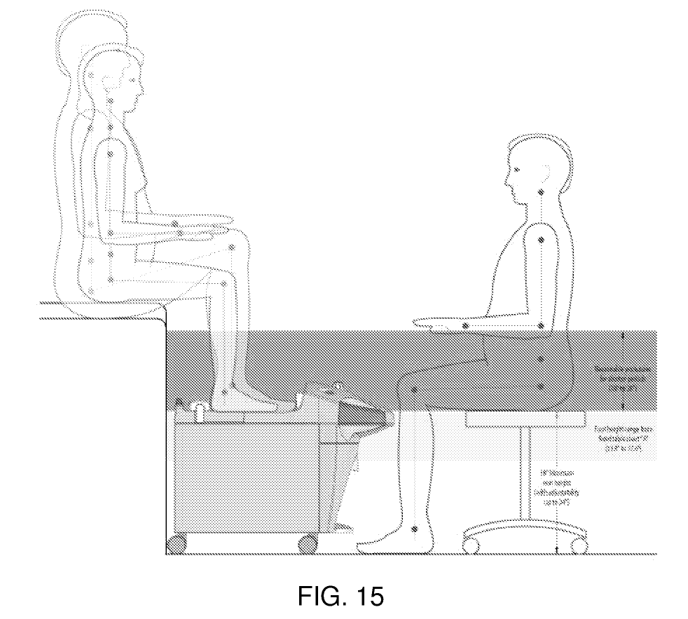

In accordance with certain examples, the device and methods disclosed herein may be integrated into a system that is configured to provide treatment. The system may be used in an office setting of a medical practitioner, e.g., physician, podiatrist, etc., or may be configured for use in the home. One example of a system configured for use in an office setting for treatment of skin disorders, e.g., nail infections, is shown in FIGS. 14A and 14B. The system 1400 includes a housing 1410 which contains the electromagnetic energy source, controller and associated circuitry. The housing 1410 is positioned on a set of wheels or casters 1412, 1414, 1416 and 1418 to facilitate easy movement of the system 1400 from place to place. The housing 1410 includes a locking pedal 1420 to prevent or retard movement of the system 1400 once positioned. The housing 1410 also includes a retractable roller handle 1425 and positioning handles 1426 and 1427 to facilitate movement of the system 1400. The system may include a storage drawer 1429. The system 1400 shown in FIGS. 14A and 14B is configured for treatment of tissues on the foot, e.g., nail tissue, skin and the like. The system 1400 includes a heel retainer 1430 and a foot platform 1440 for placement of a subject's foot as shown in FIG. 15. The heel retainer 1430 is mounted on bar 1432 which can slide along the surface of foot platform 1440 and aid in positioning the foot on the foot platform 1440. The system 1400 also includes a safety shut off switch 1450 and a touch-screen interface 1460 that may display temperature of a temperature sensor (not shown). The applicator 1470 is positioned such that placement of the foot on the foot platform and heel retained brings the portion of the foot to be treated in contact with the applicator 1470. The applicator 1470 may be configured for movement horizontally, e.g., perpendicular to bar 1432, such that treatment of different areas of the foot may be accomplished without having to reposition the foot. The applicator 1400 may also be configured for vertical movement to account for differences in foot thickness of different subjects. The applicator 1470 may take any of the configurations disclosed herein depending on the type of electromagnetic energy that the apparatus 1400 is designed to provide to the tissue.

In accordance with certain examples, the systems disclosed herein may be configured to deactivate or kill an organism infecting a nail. Organisms that are known to infect the nails include, but is not limited to, Epidermophyoton floccosum, Trichophyton rubrum, Trichophyton mentagrophytes, Candida albicans, Aspergillus, Acremonium, Fusarium, Scopulariopsis, Scytalidium, and Hendersonula toruloidea. In one example, a device that includes an ultraviolet, visible or infrared light energy source coupled to an applicator is disclosed. In some embodiments, the wavelength of the energy is greater than about 200 nm, more particular greater than about 340 nm, e.g., greater than about 400 nm.

In certain examples, the energy is provided to the nail in either a continuous or pulsed form. In examples where continuous exposure is implemented, a light source such as an arc lamp or mercury lamp may be coupled to the applicator. In examples where pulsed exposure is implemented, a pulsed laser may be used. The pulse rate of the laser may be controlled, for example, through a controller or processor. Illustrative pulsing frequencies include, but are not limited to, 0.1-30 Hz, more particularly about 0.1 to 10 Hz, e.g., about 1 Hz, or about 10-20 Hz, e.g., about 15 Hz. In embodiments where a laser is used, the pulse width of the laser may vary, for example, from about 5 μseconds to about 30 seconds, more particularly about 50 μseconds to about 10 seconds, e.g., about 100 μseconds to 1 second or about 1 millisecond, 5 milliseconds, 10 milliseconds, 50 milliseconds, 100 milliseconds, 500 milliseconds or other selected pulse widths within the illustrative ranges disclosed herein. Illustrative lasers include, but are not limited to, pulsed dye lasers, a nitrogen gas laser, an excimer chemical laser, a Nd:YLF laser, a Nd:YAG laser, a frequency doubled Nd:YAG laser, a Nd:glass laser, a copper vapor laser, an alexandrite laser, a frequency doubled alexandrite laser, a titanium sapphire laser, a ruby laser, a fiber laser, a diode lasers, a helium-neon gas laser, an argon ion gas laser, a krypton ion gas laser, a xenon ion gas laser, a carbon dioxide gas laser, a carbon monoxide gas laser, an HF laser, a DF laser, a chemical-oxygen iodine laser, a HeCd metal vapor laser, a HeHg metal vapor laser, a HeSe metal vapor laser, a copper vapor laser, a gold vapor laser, an Er:YAG laser, a Nd:YVO laser, a Tm:YAG laser, a Yb:YAG laser, a Ho:YAG laser, a vertical cavity surface emitting laser (VCSEL), a free electron laser, a Raman laser or other suitable lasers having at least one wavelength in the X-ray, ultraviolet, visible or infrared regions.

In accordance with certain examples, the electromagnetic energy source may be contained within a housing having an opening or aperture to transmit the beam of radiation to the target area. In certain embodiments, the applicator may be coupled to a source to direct electromagnetic energy to the target area of the nail tissue. In some examples, the system may include a light guide positioned relative to the nail plate. The light guide may be operative to couple the beam of radiation to the diseased nail. A sensor may be used to determine when sufficient thermal energy has been delivered to the target area to thermally deactivate the unwanted organism. The sensor may be, for example, a photodetector (e.g., an IR detector) or a temperature sensor. A controller or processor may be used to deactivate the source should any adverse effects occur during treatment, e.g., a patient becoming uncomfortable.

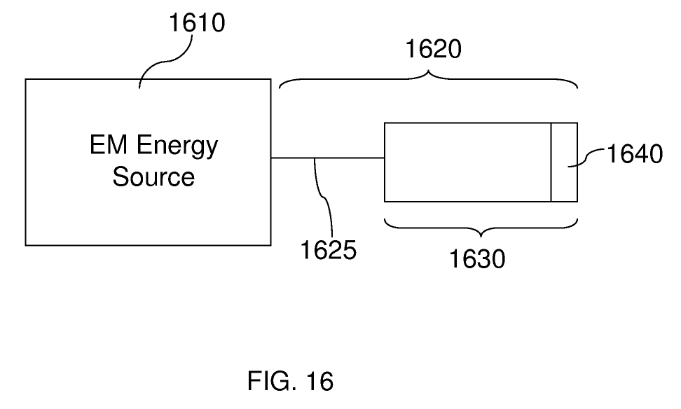

In accordance with certain examples, an illustrative system for delivering electromagnetic energy to an infected nail is shown in FIG. 16. The system 1600 includes an electromagnetic energy source 1610 and an applicator 1620. The energy source 1610 is typically contained within an enclosure or housing as discussed elsewhere herein. The housing may include an aperture or opening for transmission of the energy to the applicator 1620 and to a target area to be treated. For example, a beam of energy provided from energy source 1610 may be directed to a target area of a nail, nail plate or nail bed using applicator 1620. Many different configurations for the applicator 1620 are possible and any configuration may be used so long as some portion of the light is passed from the energy source 1610 to the applicator 1620. In one embodiment, the applicator may include a fiber 1625 with a selected cross-section (e.g., circular) and an adaptor or guide 1630 for directing the light. The adaptor 1630 may include optics such as lenses, filters and the like to provide light having desired properties, e.g., polarized, filtered, etc. The adaptor 1630 may be placed in direct contact with the nail or may be placed above or beside the nail. The adaptor may optionally include a removable spacer 1640 to keep the adaptor a fixed or selected distance from the nail to be treated.

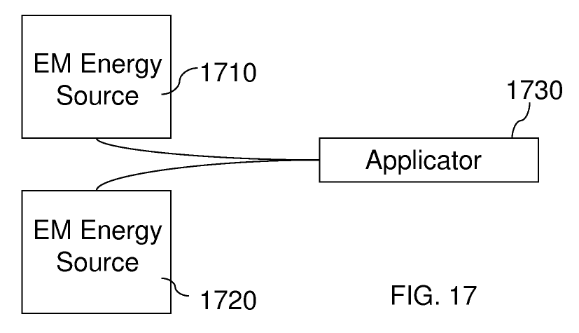

In accordance with certain examples, the exact configuration of the electromagnetic energy source 1610 may vary depending on the type of energy to be delivered. In certain embodiments, the electromagnetic energy source 1610 is a coherent or an incoherent light source, a microwave generator, a sound wave generator, a radio frequency generator or the like. In certain embodiments, an electromagnetic energy source configured to deliver ultrasonic energy to the nail may be used. In some embodiments, two or more different energy sources may be used. For example and referring to FIG. 17, a first electromagnetic energy source 1710, e.g., a microwave generator, may be energetically coupled to an applicator 1730. A second electromagnetic energy source 1720, e.g., a radio frequency generator, may also be coupled to the applicator 1730. A controller (not shown) may be used to control which energy source provided energy to the applicator 1730. Alternatively, the first and second electromagnetic energy sources may provide energy simultaneously. For example, one of the energy sources may provide an incoherent light beam to the applicator while the second source may provide a coherent light beam to the applicator. Other configurations using two or more sources will be readily apparent to the person of ordinary skill in the art, given the benefit of this disclosure.

In accordance with certain examples, the duration of treatment for treating an infected nail may vary from person to person and may vary depending on the wavelength of the energy that is used. In certain examples, the wavelength is between about 200 and 400 nm. In some examples, the wavelength is between about 200 nm and 2600 nm, more particularly about 400 nm to about 1800 nm, even more particularly about 400 nm to about 1100 nm, e.g., about 1160 nm to about 1800 nm. In other examples, the wavelength may be between about 400 nm to about 700 nm, more particularly about 500-600 nm, e.g., about 585-600 nm.

In accordance with certain examples, the energy density or fluence of the electromagnetic energy source may vary depending on the configuration of the applicator, the selected electromagnetic energy source and the like. Energy also depends on the duration of treatment, e.g., energy delivered may be approximated by multiplying the power by the exposure time. In certain examples, the energy density is about 1 J/cm² to about 200 J/cm², more particularly about 1 J/cm² to about 50 J/cm², e.g., about 2-20 J/cm or 4-10 J/cm². The exact shape and size of the energy delivered to the tissue may also vary with the configuration of the applicator. In certain embodiments, the energy has a circular cross-section with a diameter of about 1 mm to about 30 mm, more particularly about 2 mm to about 20 mm, e.g., about 7-10 mm.

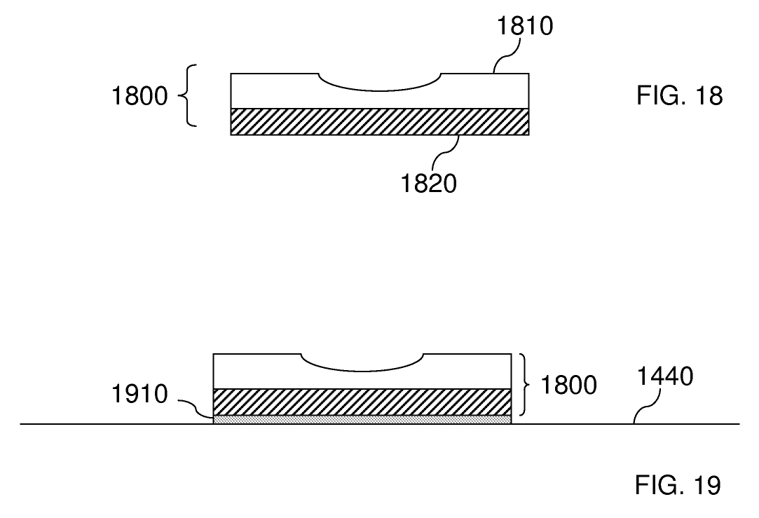

In accordance with certain examples, the system shown in FIGS. 14A and 14B may be used to treat an infected nail. Alternatively or for use with the systems shown in FIGS. 14A and 14B, a mold or insert configured to receive a toe, fingernail or the like may be used to position the nail for treatment. The mold or insert may be cast using the patient's toe or finger or may be a mold that is constructed based on the average size of people's fingers or toes. A side-view of an example of an insert is shown in FIG. 18. The insert 1800 includes a top portion 1810 in thermal communication with a base 1820. The base 1820 may be configured with an adhesive to keep the insert 1810 from moving or sliding during treatment. In the alternative, the base may be configured as a heat sink or cooling device to remove heat from the toe or finger to prevent unwanted tissue damage. The base may be configured to receive a cooling agent, such as liquid nitrogen, dry ice, a frozen gel, ice, cold water or other suitable agent that can facilitate heat transfer to the base from the finger or toe. In some examples, the base may be configured to provide impedance matching to facilitate more uniform exposure of the nail to the energy. The insert 1800 may be used with the system of FIGS. 14A and 14B by placing the insert on the foot platform 1440 as shown in FIG. 19. A thin layer of adhesive 1910 may be placed between the foot platform 1440 and the insert 1800 to prevent or retard movement of the insert 1800 during treatment. In certain examples, the container configured to receive a bolus 530 may be shaped similar to the insert 1800 such that proper positioning of the toe or finger is further facilitated.

In accordance with certain examples, the electromagnetic energy may be delivered to any portion of the nail. In some examples, the electromagnetic energy is delivered to one or more of the nail plate, the cuticle, the nail bed, or the nail root. In certain embodiments, the applicator may be positioned to first treat the nail bed and then move or be moved to treat some other portion of the nail, e.g., the nail root (which is typically called the nail matrix), cuticle or nail plate. In some embodiments, the width of the beam may be large enough to treat all areas of a nail simultaneously.

In accordance with certain examples, one or more naturally occurring agents in the nail or skin may be used to enhance treatment. For example, molecules in the nail itself may include, but are not limited to, a blood vessel, a wall of a blood vessel, melanin, water, collagen, a red blood cell, a white blood cell, hemoglobin, plasma, interstitial fluid, intracellular fluid, the disease causing organism, or any combination thereof. Energy may intentionally be used to cause absorption by the species in the nail, or the species in the nail may absorb energy incidental to the energy delivered for treatment.

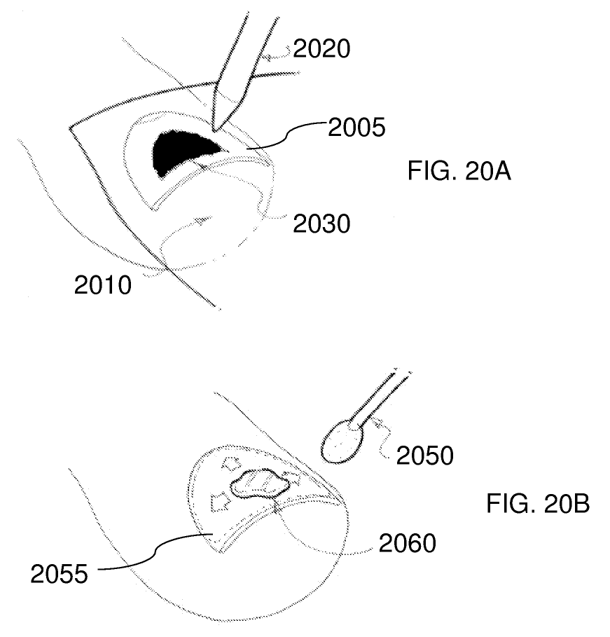

In accordance with certain examples, one or more agents may be coated or otherwise disposed on the nail prior to treatment. Illustrative agents include, but are not limited to, dyes, chromophores, radiation absorption agents, metallic paints and therapeutics. These agents may be applied to absorb the electromagnetic energy to aid in treatment or may be used to absorb the electromagnetic energy to prevent exposure of certain tissues to the energy. Referring to FIG. 20A, an agent may be impregnated in a transfer sheet 2010 and transferred to a nail 2005 by placing the transfer sheet on the nail 2005 and applying pressure to the transfer sheet with device 2020 to provide a coating 2030 on the nail 2005. Device 2020 may be any suitable device that can apply pressure including, for example, a stylus, pen, metal, cotton swab, or plastic rod or the like. In another example, the agent may be coated on the nail by applying the agent with a cotton swab. For example and referring to FIG. 20B, a cotton swab 2050 may be used to dispose a coating of an agent 2060 on a nail 2055.

In certain examples, a therapeutic in combination with another agent may be coated or added to the nail prior to treatment. For example, one or more anti-fungals or anti-bacterials may be mixed with the agent and the mixture may be coated or otherwise disposed on the nail. In the alternative, an anti-fungal or anti-bacterial agent may be chemically linked to the agent and resulting composition may be disposed on the nail. Additional methods for applying therapeutics in combination with another agent will be readily selected by the person of ordinary skill in the art, given the benefit of this disclosure. In some examples, one or more cosmetic agent may be applied post-treatment to improve the appearance of the tissue. Illustrative cosmetic agents include, but are not limited to, ELON™ Complex 38, Mavala Ridge Filler and Nail Tek Foundation II Ridge Filler.

In accordance with certain examples, the energy delivered to the nail may be selected to traverse the nail plate and be absorbed by the nail bed and/or the organism infecting the nail to reduce heating of the nail plate. As the nail bed and nail plate absorb heat, it may remain heated for an extended period of time, which can lead to unwanted injury to the surrounding tissue. Tissue injury depends on temperature and on the time at the elevated temperature. In certain examples, the tissue may be heated to between about 40° C. and about 80° C., more particularly about 43-70° C., e.g., about 50° C. or 55° C. In certain examples, the organism infecting the nail is heated to an effective temperature to either deactivate the organism or to kill the organism while keeping the temperature of the nail tissue below an acceptable level to avoid permanent tissue damage, e.g., permanent tissue damage to the nail bed.

In accordance with certain examples, the energy may be delivered to the nail to treat the entire nail at once or may be delivered as a focused beam to treat only a portion of the nail at a time. When the energy is delivered as a focused beam, it may be desirable to move the applicator to different areas on the nail to provide for more effective treatment of the nail. This movement may be done manually by the medical practitioner or may be automated using a motor, robotic arm or other devices that may be attached to the applicator and can effectuate movement. A map of the nail may be made and stored in a computer system, and the motor may be computer controlled to move the applicator over substantially all surfaces of the nail.

In accordance with certain examples, one or more channels or holes may be drilled or otherwise made in the nail to facilitate delivery of the electromagnetic energy, optionally in combination with agents such as therapeutics, to tissue underlying the nail. These channels or holes may be drilled to minimize absorption of the electromagnetic energy by the nail itself. In one embodiment, a sample of the organism may be taken to determine a wavelength of energy at which the organism will absorb. The organism may be viewed under a microscope, e.g., with or without stain, or spores produced by the organism may be used to assist in the identification of the infectious organism. Many organisms infecting the nail, e.g., the dermatophytes discussed herein, are observed to be an orange/brown color. By selecting a wavelength of about 400-550 nm, the operator can increase the amount of energy absorbed by the organism. In other examples, the entire nail may be removed and the underlying tissue may be treated with a selected electromagnetic energy to deactivate or kill any remaining infectious organisms.

In accordance with certain examples, the electromagnetic energy delivered to the nail may be microwaves or radio waves or the energy may take other forms, such as sound waves. For example, the energy source may be a radio frequency generator or a microwave generator to produce heat within a diseased nail to deactivate or kill the organism. It is believed that the infectious organism absorbs the microwaves, or radio frequencies to a greater extent than the nail tissue which results in heating or superheating of the organism and eventual deactivation or killing of the organism. It may be desirable to capacitively couple the applicator with the nail. In one embodiment, an adaptor that substantially covers the entire surface of the nail may be used optionally with a tuning box and a bolus as discussed elsewhere herein. In one example, the frequency used to treat the nail is greater than about 100 kHz, more particularly greater than about 1 MHz, e.g., about 10 MHz or more or 300 MHz or more.

In certain examples, the energy source may be an sound generator such as, for example, a high intensity ultrasound source or a high power focused ultrasound source. Sound waves generated by the ultrasound generator may be used to heat the infectious organism to deactivate or kill the organism. An applicator configured to deliver sound waves may be impedance matched or impedance mismatched depending on the desired results of the treatment. Impedance mismatching of the applicator and the nail may be desirable, for example, to selectively target absorption of the sound waves by the organism rather than the nail.

In accordance with certain examples, the exact frequency of the treatment protocol for the nail depends, at least in part, on the degree of infection, the temperature used and the like. In certain examples, treatment of the nail occurs daily, weekly, bi-weekly, monthly, semi-monthly, once every three months, once every six months or once per year. In embodiments where treatment is performed for prophylactic reasons, e.g., to prevent reoccurrence of the infection, treatment may be performed less frequently than treatment for an active infection. Also, as discussed in more detail below, by taking an immediate culture of the infectious organisms, efficacy of the treatment may be monitored more rapidly than is possible with existing oral administration of therapeutics.

In accordance with certain examples, many different types of adaptors may be used to provide electromagnetic energy to the nail. These adaptors may be single use, e.g., disposable, or may be configured for multiple uses. In the configuration where the adaptor is designed for multiple uses, the adaptor may be constructed of suitable materials that can withstand chemical treatment and or sterilization equipment, such as an autoclave. In examples where the adaptor is configured for a single-use, the adaptor may be a conductive or non-conductive material that has sufficient strength for at least the treatment period.

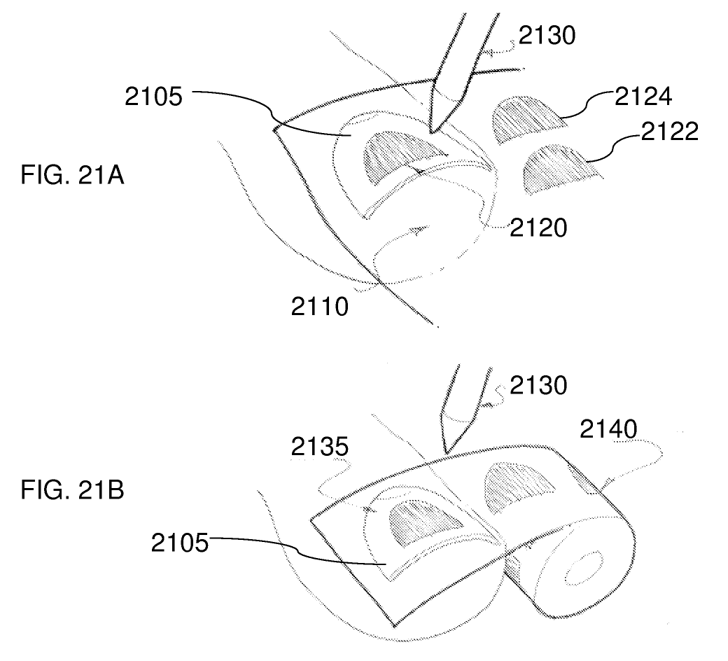

In certain examples, a sheet of metal or other conductive material may be used to dispose an applicator on a nail. An example of this is shown in FIGS. 21A and 21B. This process is similar to the transfer sheet used to dispose an agent on a nail. Referring to FIG. 21A, a transfer sheet 2110 may be placed on the nail 2105. The transfer sheet 2110 includes patterns 2120, 2122, 2124, which are geometrically similar to the shape of the nail 2105 but are of different sizes. In use, a pattern having a size that closely matches the size of the nail 2105 is placed over the nail 2105, and device 2130, e.g., a stylus, applies pressure to the pattern to transfer material from the transfer sheet 2130 to the nail 2105. Transfer of material provides a conductive coating that can be electrically coupled to the applicator for treatment. In another embodiment using a transfer sheet (FIG. 21B), the transfer sheet may be configured as a roll 2140. The roll 2140 may include different patterns which can be transferred using device 2130 to form a coating 2135 on the nail 2105.

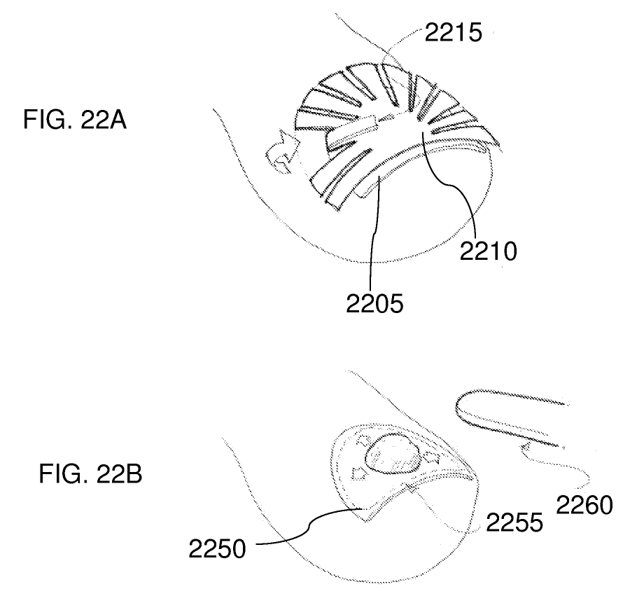

In other examples, an adaptor may be created by placing a conductive plate having arms or strips over the nail as shown in FIG. 22A. The conductive plate 2210 may be placed on the nail 2205 and the arms may be folded back to provide a shape that conforms to the shape of the nail. For example, an arm 2215 is shown as having been folded back in FIG. 22A to the edge of the nail. The conductive plate may be electrically coupled to applicator prior to treatment.

In another example, a conductive material such as a putty or gel may be disposed on the nail as shown in FIG. 22B. The conductive material 2255 may be disposed on the nail 2250 using a swab, dropper, by hand or the like. An applicator 2260 may be electrically coupled to the conductive material 2255 by placing the applicator on top of the disposed conductive material 2255. The conductive material 2255 may be tacky to retain the applicator 2260 for a sufficient period to allow for treatment. For example, a gel or a putty may be used to provide a smooth surface over an irregularly-shaped or dismorphic tissue, e.g., a disphormic nail plate or nail bed. In addition, a material may be used to a large applicator, e.g., one larger than the target tissue area, to provide a pathway for heat transfer (EM waves) through to only the target area.

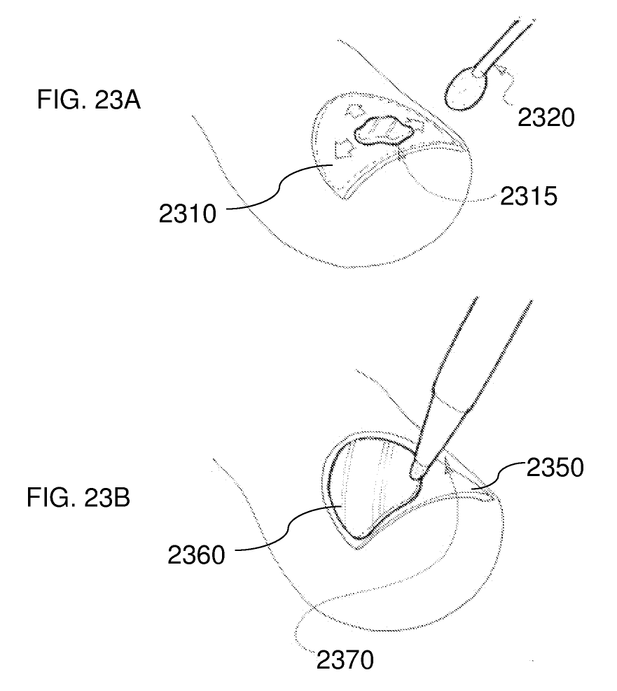

In another embodiment, the conductive material may be painted on the nail or otherwise disposed on the nail. For example and referring to FIG. 23A, conductive material 2315 may be disposed on the nail with a cotton swab 2320, or similar device, by tracing the nail 2310 with the cotton swab 2320. The disposed conductive material 2315 may be electrically coupled to an applicator for treatment. In another example, the conductive material may be loaded in a paint pen, or comparable device, and applied to the nail. For example, and referring to FIG. 23B, paint pen 2370 may be used to dispose conductive material 2360 on nail 2350.

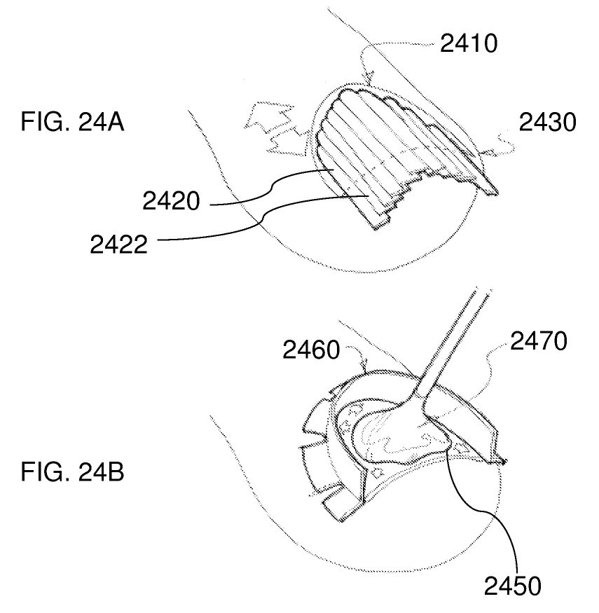

In another example, conductive strips may be disposed on the nail. For example and referring to FIG. 24A, metal strips, such as metal strips 2420 and 2422 may be disposed on nail 2410. The ends of the conductive strips, shown at dotted line 2430, may be trimmed away prior to treatment to provide an adaptor of conductive strips that covers the nail. At least one of the conductive strips may be electrically coupled to the applicator for treatment of the nail 2410. In another embodiment, a form or mold may be used to dispose a conductive material on the nail. Referring to FIG. 24B, a form 2460 is placed on the nail 2450 and is configured to rest around the edge of the nail surface. A conductive material 2470 may be disposed on the nail 2450 and flow or move into the space of the mold 2460. Once set up or cured, the mold 2460 may be removed and the conductive material 2470 may be electrically coupled to an applicator for treatment of the nail 2450.

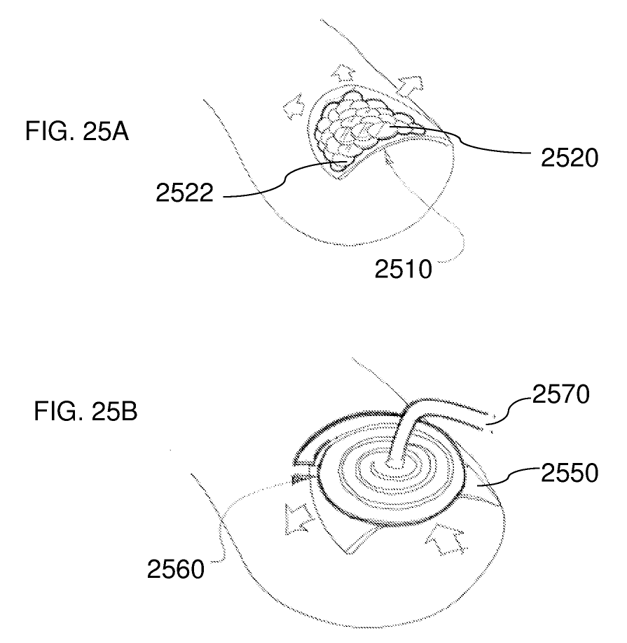

In another embodiment, individual conductive elements may be placed on a nail in a sufficient amount and with suitable spacing to cover the nail surface. For example and referring to FIG. 25A, a series of small conductive circles, such as circles 2520 and 2522, have been disposed on a nail 2510 in a sufficient amount to cover the entire nail surface. While shown as circles in FIG. 25A, other shapes, e.g., square, rectangular, triangular, etc., may be used and in some examples it may be desirable to use many different types of shapes to cover the nail surface, e.g., circles in combination with triangles. Once the nail surface is covered, one or more of the conductive elements may be electrically coupled to an applicator to provide treatment to nail 2510. In another example, a coil of conductive material may be placed on the nail. For example and referring to FIG. 25B, a coil 2560 of conductive material may be placed on a nail 2550. The coil 2560 is electrically coupled to an applicator through interconnect or cable 2570. Electromagnetic energy may be delivered to the coil 2560 for treatment of the nail 2550. A layer of adhesive or a conductive material may be placed between the nail 2550 and the coil 2560 to enhance treatment even further.

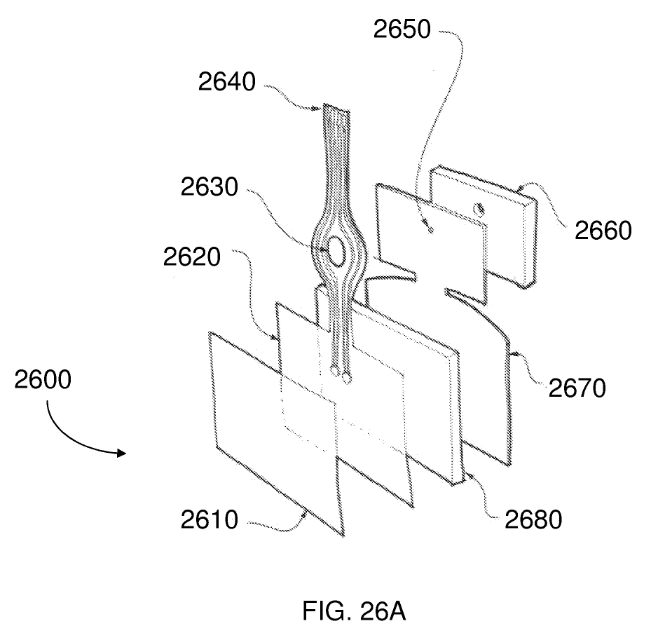

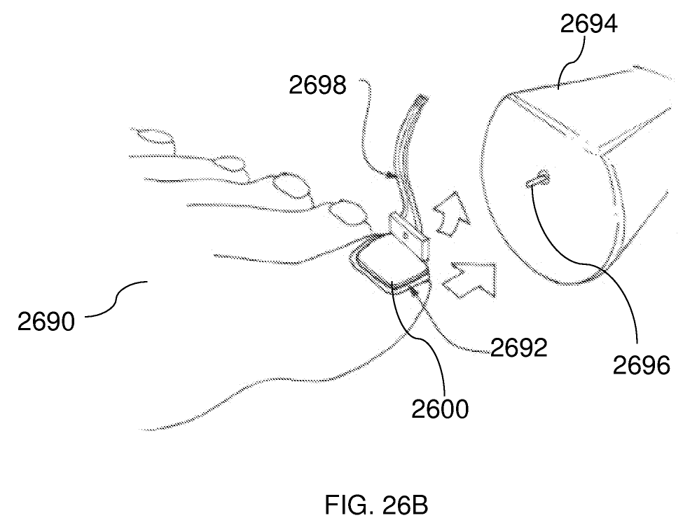

In accordance with certain examples, the adaptor may be configured as a multi-layer structure. For example and referring to FIG. 26A, an adaptor 2600 includes an adhesive backing 2610, an adhesive flex-circuit 2620 with surface-mounted thermistors and copper traces 2630 and having a plug end 2640, an adhesive pad 2680, a copper sheet 2670 with a hole 2650 and a copper block 2660. The copper block 2660 may be attached using solder, may be diffusion bonded or may be attached to the copper sheet using other methods or materials. In operation, a trace of the toe may be performed by placing a piece of clear tape on the toe and tracing the shape of the nail bed area, which may or may not be covered all or in part by nail plate. The clear tape may be transferred to the adaptor 2600, which may be trimmed to the traced shape. The trimmed adaptor may then be placed on a nail bed area and the copper block 2660 may be bent up to change the angle of the copper sheet 2670 and the flex circuit 2620. For example and referring to FIG. 26B, apparatus 2600 has been placed to cover the nail bed area, which may or may not be covered all or in part by nail plate, on the big toe of foot 2690. The apparatus has been positioned on the nail bed area such that it sits about 1 mm away from the exposed skin on the big toe, as pointed out by arrow 2692. The copper sheet 2670 and copper block 2660 may be coupled to a pin 2696 in applicator 2694, as shown in FIG. 26B. The plug end 2640 of the flex circuit 2620 may be electrically coupled to interconnect 2698 and treatment may be initiated.

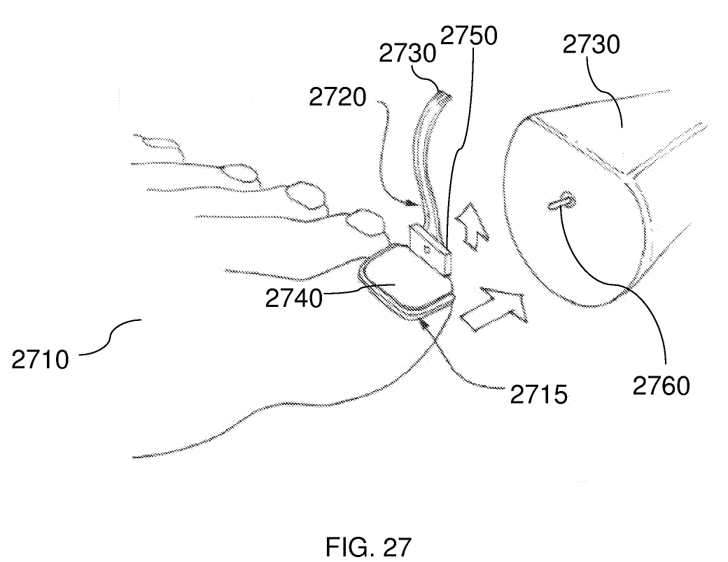

In other configurations, the adaptor 2600 may be trimmed such that it overlies the entire nail bed area and exceeds the nail bed area by about 1 mm on all sides. An example of this configuration is shown in FIG. 27, where adaptor 2715 has been placed over the nail bed area of the big toe of foot 2710. The adaptor is slightly larger than the shape of the nail bed area of the big toe, e.g., 1-2 mm larger on all sides, as pointed out by arrow 2715. The copper sheet 2740 and copper block 2750 may be coupled to pin 2760 in applicator 2730. The flex circuit 2720 may be electrically coupled to interconnect 2730 and treatment may begin. While the illustrative examples shown in FIGS. 26A, 26B and 27 were described in reference to a trace of the toe with tape to trim the adaptor to size, the tape trace of the toe may be omitted. Instead, the adaptor may be placed on and/or beyond the nail bed area of the toe and a trace of the nail bed area of the toe may be performed on the adaptor itself. The adaptor may be removed and trimmed to size and placed back on the nail bed area of the toe. The adaptor may be electrically coupled to an applicator and treatment may be initiated. In examples where a tape trace is omitted, each layer of the adaptor may be trimmed, e.g., the adhesive backing, copper sheet and copper block may be trimmed, or one or more layers of the adaptor may be left untrimmed. For example, it may be desirable to leave the adhesive backing whole to provide better contact with the surface of the nail, while the copper sheet and copper layer may be trimmed to size. Additional configurations that use a flex circuit will be readily apparent to the person of ordinary skill in the art, given the benefit of this disclosure.

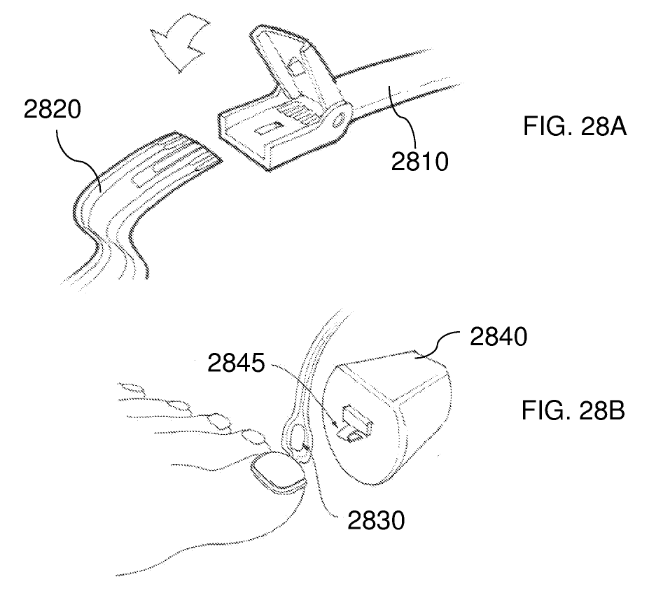

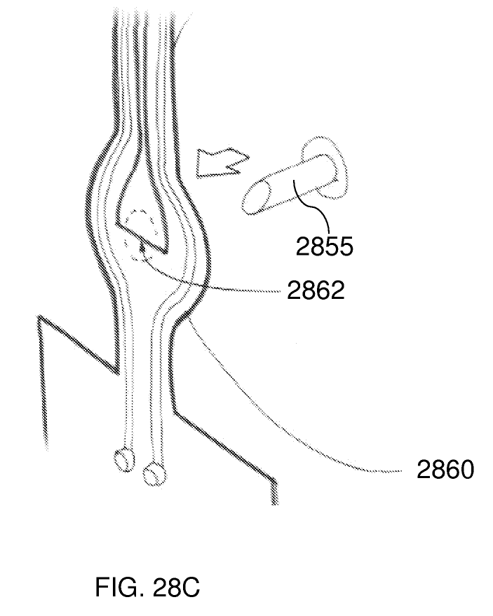

In accordance with certain examples, the adaptors or applicators disclosed herein may be configured with one or more features that render them usable only once. For example and referring to FIG. 28A, a connector 2810 may be used such that the flex circuit 2820 is electrically coupled to the applicator. Opening of the connector 2810 results in breaking of the electrical connection, which cannot be restored by closing the connector 2810. As the flex circuit is an integral part of the adaptor, the adaptor is not capable of being re-used. Such single use adaptors reduce the likelihood of cross-contamination. Another example of a single use adaptor is shown in FIG. 28B. In this illustration, the adaptor 2830 is electrically coupled to the applicator 2840 through a spring 2845. The spring 2845 may be inserted in the hole in the adaptor 2830 and onto the surface of adaptor 2830 to provide electrical contact between the applicator 2840 and the adaptor 2830. When the spring 2845 is removed, the adaptor is damaged so that it may not be re-used again. Another embodiment of a single-use adaptor is shown in FIG. 28C. The applicator includes a post or projection 2855 that punctures adaptor 2860 at area 2862 during removal of the adaptor 2860 from the applicator. This puncture prevents electrical coupling of the adaptor to the applicator. Other configurations and features that render an adaptor suitable for only a single use will be readily selected by the person of ordinary skill in the art, given the benefit of this disclosure.

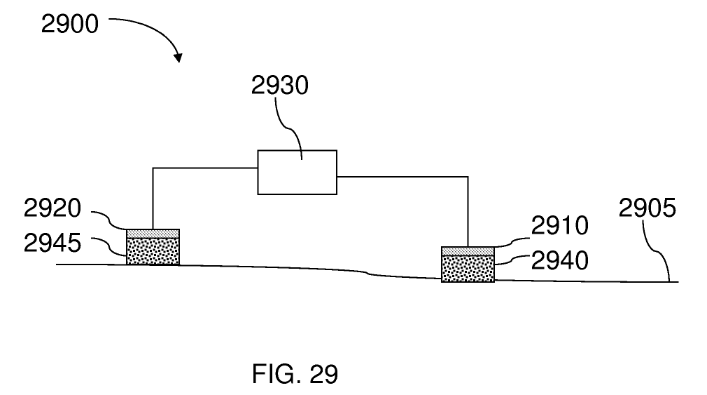

In accordance with certain examples, iontophoretic or electrokinetic delivery of a compound may be used in combination with the devices, systems and methods disclosed herein to deliver a therapeutic to the tissue. In some examples, the adaptor may be configured for electromagnetic energy delivery and for iontophoresis or electrokinetic delivery of a compound, such as a therapeutic. While not wishing to be bound by any particular theory or this example, iontophoresis is a process whereby a compound is introduced into a tissue or a cell by application of an electric field. Electrokinetic delivery involves iontophoresis and also involves electroosmosis. Electroosmosis is the bulk fluid flow associated with ion transport by an electric field. An illustrative device for iontophoresis or electrokinetic delivery of drugs is shown in FIG. 29. The device 2900 includes a cathode 2910, an anode 2920 each connected to a power supply 2930 (which may be a DC or an AC power supply). The cathode 2910 and the anode 2920 are configured as plate electrodes in FIG. 29, though other configurations are possible. The anode 2920 and cathode 2910 are coupled with the tissue 2905 through carriers 2945 and 2940, respectively. Carrier 2945 typically includes a compound to be delivered to the tissue 2905, whereas carrier 2940 typically includes a saline solution or some other salt solution. During operation, electrons flow from the cathode to the anode and the electric field drives the negatively-charged compound from the carrier 2945 and into the tissue 2905. In the situation where an alternating current source is used, suitable circuitry may be implemented to drive the compound into the tissue. Illustrative devices and circuitry may be found, for example, in U.S. Pat. No. 7,127,285.

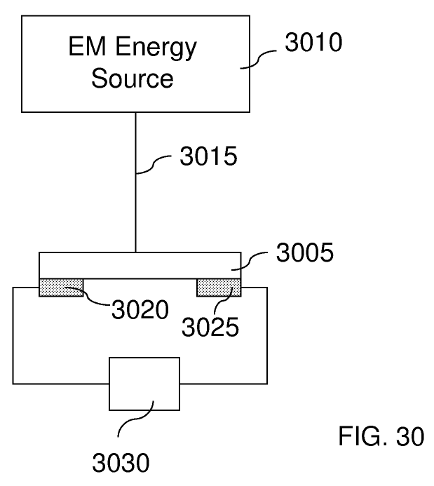

In accordance with certain examples, the iontophoresis or electrokinetic delivery device may be integrated with the adaptors disclosed herein. For example and referring to FIG. 30, an adaptor includes a metal plate 3005 configured to deliver electromagnetic energy from electromagnetic energy source 3010 through cable 3015 and to a tissue, e.g., to deliver microwaves to a tissue. The adaptor also includes a first electrode 3020 and a second electrode 3025 connected to a power supply 3030. In operation, the electrodes 3020 and 3025 rest atop a carrier that is contact with the tissue. As current is applied to the electrodes 3020 and 3025, a therapeutic in the carrier may be delivered to the tissue or delivered to an area near the tissue. Electromagnetic energy may be simultaneously delivered to the tissue or may be delivered to the tissue before or after the therapeutic is delivered. In some examples, iontophoresis or electrokinetic delivery is used to deliver an agent that is taken up by the infectious organism and that absorbs the electromagnetic energy. This uptake followed by application of electromagnetic energy results in additional heating or superheating of the organism to deactivate or kill the organism.

In accordance with certain examples, the nature of the compound delivered to the tissue depends at least in part on the organism infecting the tissue. In certain examples, the compound may be an antibiotic, an anti-fungal or an antiviral such as, for example, ketoconazole, nystatin, griseofulvin, flucytosine, abacavir, adefovir, amprenavir, azidothymidine, behenyl alcohols, such as n-docosanol, Abreva®, brivudin, cidofovir, delaviridine, didanosine, doxorubican, efavirenz, famciclovir, fluorouracil, 5-FU, gancyclovir, indinavir, terbinafine HCl, Lamisil®, lamivudine, lobucavir, Lotrimin®, methotrexate, miconazole, Micatin®, nelfinavir, nevirapine, ribavirin, ritonavir, saquinavir, sorivudine, stavudine, tacrolimus, triamcinolone acetonide, trifluridine, valaciclovir, zalcitabine or combinations thereof. In some examples, the compound may be a non-steroidal anti-inflammatory drug (NSAID) such as, for example, ibuprofen or the like. In other examples, the compound may be a vitamin or co-factor such as Vitamin A, Vitamin E, Vitamin B12 or other vitamins or compounds commonly found in nutritional supplements.

In accordance with certain examples, electrophoresis or dielectrophoresis may be used with the treatment methods and devices disclosed herein. Dielectrophoresis uses a gradient of an electric field to drive uncharged molecules in the desired direction; these uncharged molecules are desirably polar, but they are not necessarily ions, as is the case with typical electrophoresis. Dielectrophoresis may be particularly useful where an agent to be delivered is polar, or has a dipole moment, but is not charged.

In accordance with certain examples, the methods and devices disclosed herein may be used to provide rapid feedback to assess the efficacy of treatment. It may take nine months or more to assess the efficacy of conventional treatment of tissues, i.e., oral administration of anti-fungals, especially where the tissue is keratinized tissue. For example, oral administration of terbinafine for three-six months or more is typically prescribed by a physician to treat onychomycosis. The efficacy of such treatment cannot be assessed until the nail grows out, which can take nine months or more. In the methods disclosed herein, subsequent to treatment, a microbiological culture may be obtained to assess the effectiveness of the treatment. In situations where treatment is effective, fewer or no microbiological colonies will be observed as compared to a control value. In situations where treatment is ineffective, the number of microbiological colonies will be similar to those observed in a control sample. In cases where treatment is ineffective, the patient may return for subsequent treatment or may be placed on a different type of treatment. By using this assessment method as feedback to assess treatment, the effectiveness of treatment may be increased and overall treatment time may be reduced. In certain examples, the methods disclosed herein may be used to assess whether treatment is effective within or less than one month after the first treatment. In some examples, the effectiveness of treatment may be assessed in two weeks or less. Such rapid feedback may be especially useful in the treatment of nail infections where nail growth may take several months.

In accordance with certain examples, a method of treating a skin or nail infection is disclosed. In certain examples, the method includes delivering electromagnetic energy to the infected skin or nail, and culturing organisms infecting the skin or nail to assess efficacy of treatment. The electromagnetic energy may be delivered using any of the devices, system and methods disclosed herein. The organisms may be cultured using conventional microbial culture techniques, such as those found in Bergey's Manual of Determinative Bacteriology. Based on the level of organisms in the culture, the efficacy of treatment may be determined with the goal of the treatment being reduction in the number of cultured organisms present or the entire elimination of the infectious organisms.

In accordance with certain examples, it may be desirable to subject the tissue to one or more pre-treatment steps. Pre-treatment steps include positioning of the tissue, sterilization of the tissue, e.g., using alcohol pads, washing of the tissue with soap, betadine or the like. In certain examples, the tissue may be debrided prior to treatment to remove any dead cells or thickened tissue (e.g. hyperkeratotic nail). In some examples, the onycholytic portion of the nail plate may be trimmed or clipped back prior to treatment. Additional pre-treatment steps will be readily selected by the person of ordinary skill in the art, given the benefit of this disclosure.

In accordance with certain examples, the methods disclosed herein may also be used to disinfect a hood or other culture transfer device. For example, one or more applicators may be placed in a laminar flow hood and switched on to deactivate or kill any organisms living in the laminar flow hood prior to performing tissue culture or cell culture in the laminar flow hood. In certain examples, the applicator may be configured for insertion in a culture vessel to sterilize the culture vessel prior to introduction of any cells.

In accordance with certain examples, a device configured to treat all infected nails simultaneously is provided. In certain examples, the device may include a plurality of applicators where each applicator is configured similar to or the same as one or more of the applicators disclosed herein, e.g., the applicator shown in FIG. 5. Each applicator may be mounted or slidably fixed to a system similar to the one shown in FIGS. 14A and 14B. In the configuration where the applicators are slidably fixed to the system, each applicator may be moved perpendicular to the foot and placed in contact with a nail and nail bed to be treated. In certain examples, the device may include two or more applicators, e.g., three, four or five applicators. Each of the applicators may function independent of the other, e.g., different energy levels may be applied, or a single energy level may be provided to each applicator. In certain examples, a first applicator may be configured to provide a first type of energy, e.g., ultraviolet light, and a second applicator may be configured to provide a second type of energy, e.g., microwaves. The person of ordinary skill in the art, given the benefit of this disclosure, will be able to design systems that include multiple applicators.

In accordance with certain examples, a device sized and arranged to treat the hooves of a non-human mammal is provided. In certain examples, the non-human mammal is a horse or a sheep. In certain embodiments, the applicator may be sized and arranged to treat the entire hoof of the non-human mammal, e.g., the hoof may be placed on or in an applicator that provides electromagnetic energy to all surfaces of the hoof. In certain examples, the electromagnetic energy delivered to the hoof is ultraviolet, visible or infrared light, microwaves, or radio waves. Other energies may also be delivered. In some examples, a plurality of applicators may be used to provide treatment to each hoof of a non-human mammal to reduce the time the non-human mammal must remain stationary. Other configurations for treating a non-human mammal using the devices, systems and methods disclosed herein will be readily selected by the person of ordinary skill in the art, given the benefit of this disclosure.

In accordance with certain examples, a device configured to improve the appearance of a tissue and configured for use in the home is provided. To minimize overexposure of the tissue, the device typically includes a timing circuit to provide electromagnetic energy for a selected period. The period and energy level that is provided may be based on cumulative patient data such that the selected period provides treatment for the largest number of subjects. In an alternative configuration, the user may place a temperature sensor on the tissue to be treated and the treatment may be halted when the tissue reaches a treatment temperature programmed into the device. The device may be configured with safety features that prevent use of the device unless the temperature sensor is placed on the tissue, e.g., the skin. The device may be configured to operate off of 110V power and may include cooling features such as a fan, heat sink or the like. The device may be configured to use any form of electromagnetic energy disclosed herein, e.g., ultraviolet, visible, infrared, microwaves, radio waves, etc. The device may include an on/off indicator, a safety shut off switch and the like.

In accordance with certain examples, a kit comprising at least one adaptor, a bolus and instructions for using the adaptor and the bolus is provided. In certain examples, the adaptor may be any of the adaptors disclosed herein. In some examples, the adaptor may be part of an applicator which is included in the kit. In certain examples, the bolus of the kit may be selected to provide impedance matching of the tissue and the adaptor for more uniform delivery of electromagnetic energy to the tissue. The instructions included in the kit may include any of the illustrative protocols discussed herein or other suitable protocols that may be used with the devices and methods disclosed herein.

Several specific examples are disclosed below to facilitate a better understanding of the technology described herein.

### EXAMPLE 1

An applicator for use in treating tissue of the foot or hand was constructed as follows:

In this Example, the applicator consisted of a modified coaxial cable, a tuning box, and an end-cap. The modified coaxial cable consisted of an aluminum tube of inner diameter 1.00″ and outer diameter 1.25″ (MSC Industrial Supply Co.). A 1″ long portion of the outer conductor was removed with the removed portion forming an L-shape when viewed from the side. The inner conductor consisted of a brass rod of outer diameter 0.375″ (MSC Industrial Supply Co.). See, e.g., FIG. 6. These components are machined by standard operations (lathing, drilling, tapping) to permit the unmodified end of the coaxial cable assemble to connect to a standard female microwave N-type connector (Pasternack Enterprises, Inc).

The end modified by removal of the L-shaped piece was surrounded by a second component, the tuning box, which was a modified cone of length 1.85″ and diameter 2.68″, shown in cross-section in FIG. 6 and in perspective in FIG. 5 (part 510). In this Example, the tuning box was filled completely with water to provide a low impedance path for electric fields extending from the end cap (described below) back to the outer conductor. The tuning box was formed of Duraform PA plastic by a rapid prototyping process (Quickparts, Atlanta, Ga.). The internal structure of the tuning box was such that walls of Duraform PA plastic of thickness 0.080″ separated the internal chamber filled with water from the inner conductor and from the outer conductor. In this way, the tuning box formed a self-contained chamber filled with water that slides into place onto the modified end of the coaxial cable assembly. When in position, the bottom of the cone extends 10 mm beyond the farthest reach of the outer conductor, shown in cross-section in FIG. 6.

The endcap was the third component of the applicator. It was made by cutting copper foil of thickness 0.005″ into a rectangle of 19 mm width (medial-lateral dimension of toe nail) and 14 mm length (distal-proximal dimension of toe nail); the corners were rounded to a radius of 1 mm. The copper foil rectangle was soldered to an axial block made of brass with dimensions 0.125″ width, 0.300″ height, and 0.150″ axial length, which was soldered to a transverse block made of brass with dimensions 0.520″ width, 0.300″ height, and 0.0625″ axial length. A steel pin of diameter 0.057″ was force fitted into the transverse brass block. This assembly was part 520 (all metal parts from MSC Industrial Supply Co.). The entire assembly was plated with 0.0005″ thick tin by the conventional process.

To receive the endcap assembly, a mating receptacle (Mill-Max, Inc.) was force-fitted into the exposed end of the inner conductor, which was flush with the base of the tuning box 510. By design, the pin of the endcap assembly made a press-fit into this receptacle, so that the endcap assembly may be placed and removed with finger force, as desired.

An adhesive layer was added beneath the plated copper foil of the endcap to fix it to the toenail of a patient. The adhesive was a double thickness of a Curad Scar Therapy pad (Walgreen's drug store). One adhesive surface adhered to the foil endcap, the other adhered to the toenail, and the two non-sticking surfaces were secured to each other with a cyanoacrylate glue.

Beneath the toe of a patient was placed a bolus of high water content to distribute the electric fields more evenly (see FIG. 6). In this Example, the bolus was cut from an ultrasound gel pad (AquaFlex, Parker Laboratories) to form a quadrilateral, as shown in FIG. 6. The left side in FIG. 6 was 25 mm high, the bottom was 20 mm long, and the right side was 20 mm high; the width (dimension into page) was 20 mm. A container 530 held the bolus in place beneath the toe.

### EXAMPLE 2

A system that used the applicator of Example 1 to deliver microwave energy to a nail of the foot or hand was constructed as follows:

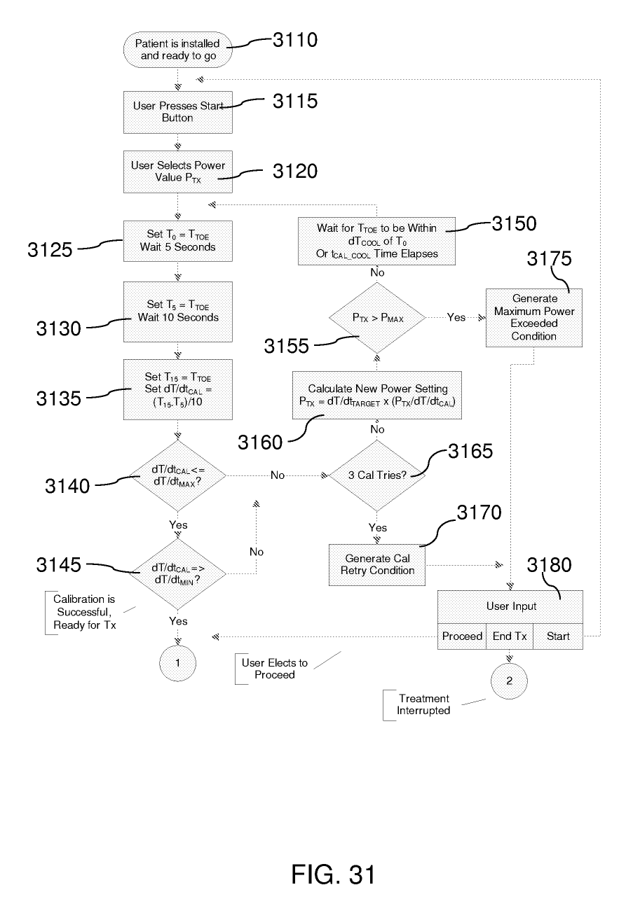

The system contained a 915 MHz, 25 Watt microwave generator that was designed and manufactured (Microwave Support Systems, Nashua, N.H.) within a 1″×10″×12″ sub-assembly housing. It was built into a metal chassis based on CAD specifications (Product Insight Acton, Mass.). The metal chassis was fabricated by a sheet metal shop, (New England Fabricated Metals, Leominster, Mass.) and the microwave energy was transmitted to an external SMA-type connector via semi-rigid copper coaxial cable. A brick of two fiber optic thermometry probes (Luxtron, Santa Clara, Calif.) resided in the chassis and were cabled to exit the chassis to be affixed to the target tissue. Also built into the system chassis was a simple micro-controller display (Mosaic Industries, Newark, Calif.) which was programmed to execute the software of the flowcharts shown in FIGS. 31A and 31B or the flowcharts shown in FIGS. 32A and 32B. The thermometry probes, the micro-controller and the microwave generator received electrical power and electrical isolation from a commercial power supply (Condor DC Power Supplies, Inc, Ventura, Calif.) that is the fourth sub-assembly in the system chassis. A custom designed power controller printed circuit board received 12V direct current from the power supply and enabled power distribution to the 5V and 12V internal circuitry.

### EXAMPLE 3

Using the applicator of Example 1 and the system of Example 2, treatment was performed on eight subjects displaying fungal infection of the large toe nail as follows. The patient's large toe nail was prepared using a double-hinged bone cuter to clip back the onycholytic nail. The clipped nail and the toe were washed with isopropyl alcohol and air dried.

To determine the maximum energy level that each subject could sustain, the following protocol was used, as shown in FIG. 31. A lower case “t” refers to a time and an upper case “T” refers to a temperature. Referring to FIG. 31, the system was calibrated by first installing the patient at step 3110. Once the patient is positioned, the start button was pressed at step 3115. A first power value PTX was selected at step 3120 by the operator. In the next several steps, the energy level was optimized. The temperature T₀ was set at step 3125 to the toe temperature Tₜₒₑ and a five second delay occurred. The temperature T₅ was set at step 3130 to the toe temperature Tₜₒₑ and a ten second delay occurs. The temperature T₁₅ is set at step 3135 to the toe temperature Tₜₒₑ. Using the temperature values at T₁₅ and T₅, a dT/dt_{cal} value was obtained by subtracting the T₅ value from the T₁₅ value and dividing by the time (10 seconds). This value represents the slope of the temperature with respect to time. A suitable operating range for dT/dt_{cal} is about 0.35 to about 0.45° C./second. The dT/dt_{cal} value also reflects how well the nail and the applicator are coupled and how much energy is being supplied. If the dT/dt_{cal} value is less than or equal to a dT/dtₘₐₓ value at step 3140 and is greater than or equal to a dT/dtₘᵢₙ value at step 3145, then the system is ready to initiate treatment. If, however, the dT/dt_{cal} value is greater than dT/dtₘₐₓ or the dT/dt_{cal} is less than dT/dtₘᵢₙ, and less than three calibration tries at step 3170 have been attempted, a new power setting 3160 is calculated and after the toe cools 3150, the calibration process is repeated beginning at step 3125. If three calibration tries have been attempted, then a calibrate retry condition may be generated at step 3170 so that user input may be prompted at step 3180. If the power exceeds a maximum power conditions at step 3175, user input may also be prompted at step 3180.

Once the system is calibrated or once the user inputs a desired power level, treatment was ready to begin. The treatment procedure used is shown in flow chart form in FIG. 32. The temperature Tₘₐₓ was first set to the target temperature T_{TARGET} at step 3202. This operation occurred either from the calibration shown in FIG. 31 or by user input at step 3204. Treatment was started at step 3206 at microwave power PTX and continued until the temperature of the toe T_{TOE} equaled or exceeded the maximum temperature Tₘₐₓ at step 3208. If T_{TOE} did not exceed Tₘₐₓ, then the setting was increased by user input at step 3210 or treatment was continued if Tₘₐₓ was not less than T_{target} at step 3212. Treatment was continued until the total treatment time tₜᵣₑₐₜₘₑₙₜ was reached at step 3214. Tₜᵣₑₐₜₘₑₙₜ was set between 5 and 20 minutes. For clarity, tₜᵣₑₐₜₘₑₙₜ is the time the microwave power was delivered to the nail. The total time from initiation of treatment to the completion of treatment was much greater than tₜᵣₑₐₜₘₑₙₜ. Once tₜᵣₑₐₜₘₑₙₜ was reached, the microwave power was turned off at step 3216 to allow the toe to cool. Once the cooling time t_{cool} at step 3218 was reached, treatment was considered complete at step 3220. If Tₘₐₓ was less than T_{target} at step 3212, then user input at step 3222 increased Tₘₐₓ by T_{adjust} at step 3224. If T_{adjust} caused Tₘₐₓ to be greater than T_{target} at step 3226, then Tₘₐₓ was set to T_{target} at step 3228 and treatment continued at step 3214. If Tₜₒₑ was not greater than or equal to Tₘₐₓ, then the sampling temperature Tₛₐₘₚₗₑ was set to Tₜₒₑ at step 3230 or 3231 and the microwave power was turned off for a time t_{hold}. When Tₘₐₓ was less than T_{target}, a user provided input at step 3234 and treatment began again. If Tₘₐₓ was greater than T_{target}, a user provided input at step 3236 to reduce the temperature by decreasing Tₛₐₘₚₗₑ by T_{adjust} 3238. In certain instances, user input at step 3240 was provided to end treatment at step 3242. In other instances, a holding time t_{hold} elapsed at step 3244. Once the holding time t_{hold} was reached, the system determined if the temperature of the toe Tₜₒₑ exceeded a maximum temperature Tₘₐₓ at step 3246. If a maximum holding time was reached t_{hold-max} at step 3248, then an error was generated and the system returned to user input at step 3204.

In the situation where Tₜₒₑ was greater than or equal to Tₘₐₓ, treatment was suspended at step 3250 for a holding time t_{hold}. After holding time t_{hold} and if Tₘₐₓ was less than T_{target} at step 3252, a user input at step 3254 was provided to increase the temperature. If Tₘₐₓ was not less than T_{target} at step 3252, then a user input at step 3256 was provided to decrease the temperature from Tₘₐₓ to Tₘₐₓ minus T_{reduce} at step 3258. After a holding time t_{hold} had elapsed at step 3260, the system determined if Tₜₒₑ was greater than Tₘₐₓ at step 3262. If Tₜₒₑ was greater than Tₘₐₓ, then the system determined if a maximum holding time t_{hold-MAX} at step 3264 had elapsed. If so, an error at step 3266 was generated and user input was required before treatment was reinitiated. If a maximum holding time t_{hold-max} at step 3264 had not elapsed, then the system returned to step 3252 and determined if Tₘₐₓ was less than T_{target}. In operations where user input was required to increase or decrease the temperature, the temperature was increased or decreased in 1° C. increments until a satisfactory result was achieved so that treatment could continue.

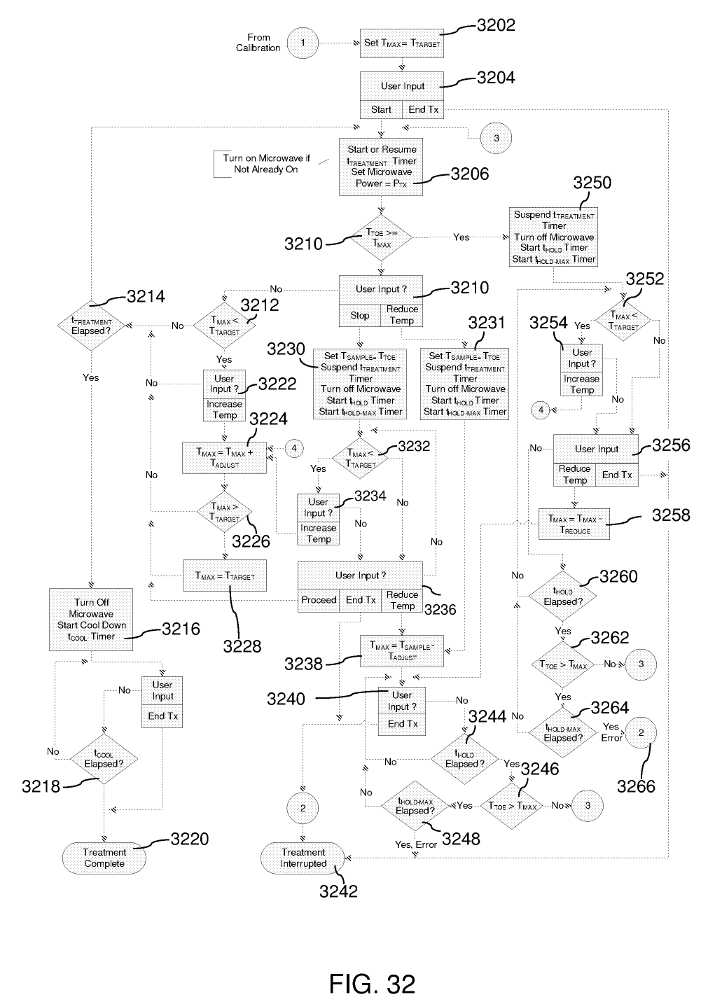

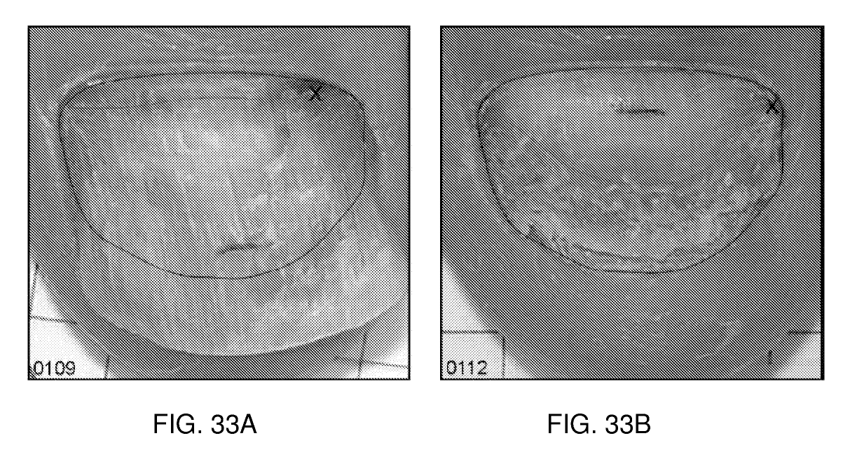

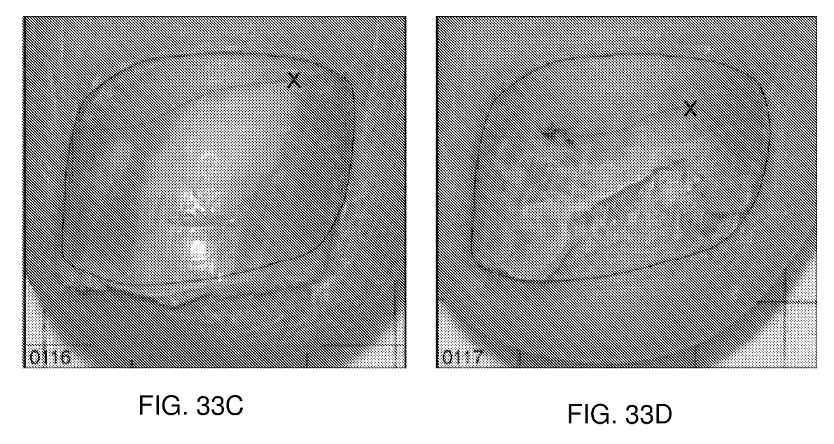

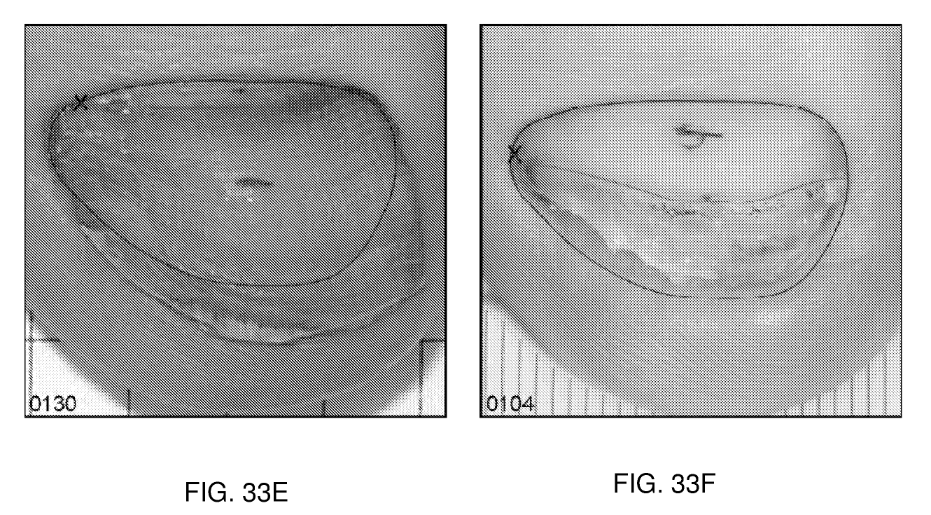

Total treatment time (the time that microwave power was applied) was between five and twenty minutes. Photographs showing the toe nail before and after treatment are shown in FIGS. 33A-33E. Referring to FIG. 33A, a fungal line of 1.6 mm from the nail bed was used as a baseline prior to any treatment. 91% of the nail was infected with the fungus prior to treatment. 3 months post treatment (FIG. 33B), the fungal line was 2.6 mm from the nail bed, and only 54% of the nail remained infected. The treatment reduced the amount of nail infected by 37%.

In another subject, a fungal line of 1.6 mm from the nail bed was used as a baseline prior to any treatment (FIG. 33C). 83% of the nail was infected with fungus prior to treatment. 4 months post treatment (FIG. 33D), the fungal line was 3.4 mm from the nail bed, and only 66% of the nail remained infected. The treatment reduced the amount of nail infected by 17%.

In another subject, a fungal line of 1.0 mm from the nail bed was used as a baseline prior to any treatment (FIG. 33E). 91% of the nail was infected with fungus prior to treatment. 5 months post treatment (FIG. 33F), the fungal line was 3.0 mm from the nail bed, and only 52% of the nail remained infected. The treatment reduced the amount of nail infected by 39%.

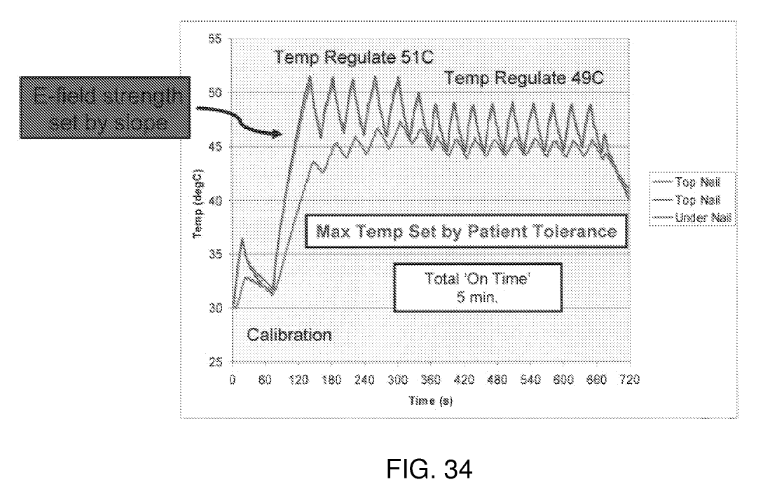

A graph showing the temperature of various portions of the nail is shown in FIG. 34. The total treatment time was five minutes and the total time for the procedure was about 720 seconds. The maximum temperature set by the subjective tolerance of the patient was 51° C. As can be seen in the graph, the tissue temperature rises and falls as the microwave power is switched on and off, respectively. This procedure allowed the temperature under the nail to fluctuate between about 47° C. and 49° C., which is believed to be a safe temperature range to avoid any permanent tissue damage. It will be recognized by the person of ordinary skill in the art, given the benefit of this disclosure, that a higher temperature range, e.g., 53-55° C., may be used depending on the tissue selected for treatment.

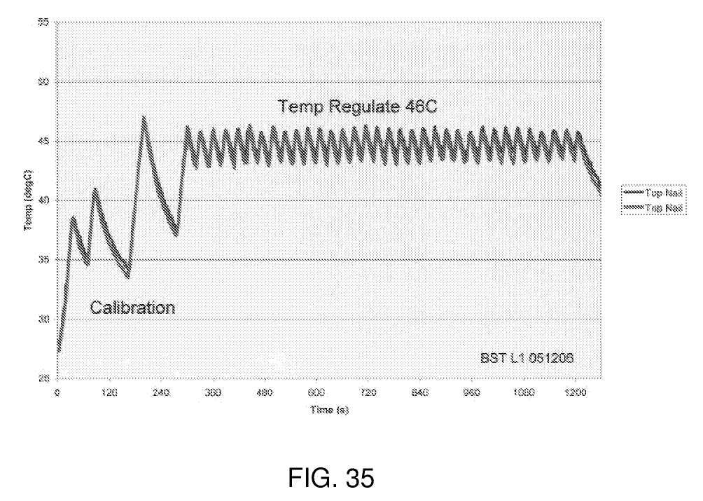

Referring now to FIG. 35, another example of a temperature profile is shown where the maximum temperature, set by patient tolerance, was 46° C. When comparing the graph of FIG. 34 with that of FIG. 35, the overall procedure time was longer when a lower Tₘₐₓ was used (1200 seconds when Tₘₐₓ was 46° C. versus 720 seconds when Tₘₐₓ was 51° C.) even though the total treatment time was five minutes in both instances.

### EXAMPLE 4

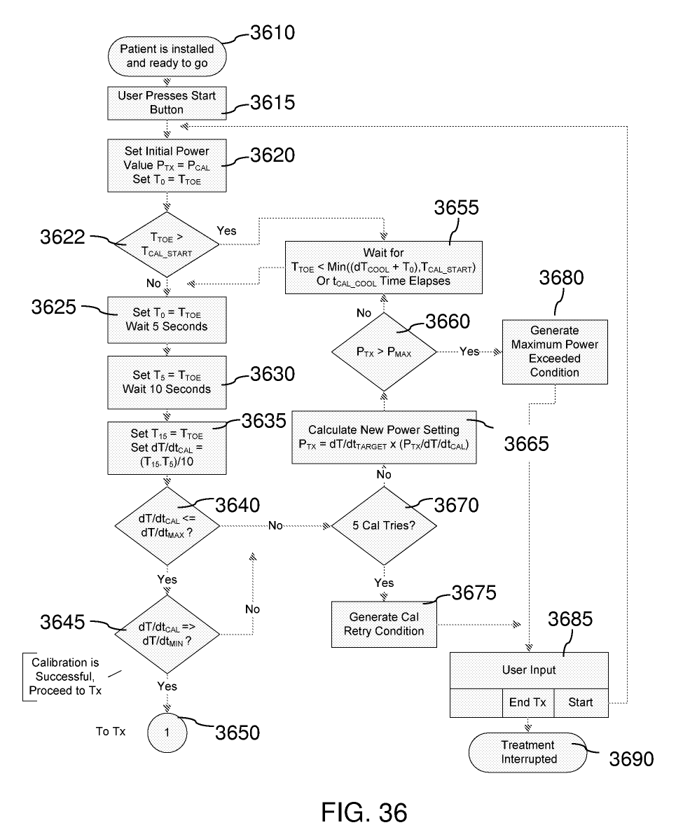

The treatment effectiveness may be assessed immediately post-treatment using any number of fungal sample analysis methods (mycological culture, dermatophyte test medium, electron microscope, etc.). Using the method of mycological culture, fungal samples were collected to screen subjects for positive culture prior to inclusion in a feasibility study. Subsequently, 10 great toes were determined to be infected with T. rubrum as confirmed by positive culture assessed by an independent mycology lab. The 10 toes were treated using the protocols described in reference to FIGS. 31 and 32, and/or FIGS. 36-38, and fungal samples were collected again immediately post-treatment. The samples were sent to the independent mycology lab for culture, incubation and assessment 60% of the samples taken were negative for fungal growth after the culture incubation period, thus providing an early indicator of the effectiveness of the treatment.

### EXAMPLE 5

A suspension of dermatophyte Trichophyton Rubrum ATCC 28188 was inoculated with human nail fragments as a nutrient source. Aliquots of this suspension were applied to a sterile filter disc and sealed in a protective envelope. A randomly selected sample of these infected discs was chosen as controls, and the rest exposed to treatment conditions using the apparatus and conditions described in Examples 1 and 2 above. 86% of infected discs treated at temperatures between 47-53° C. had no fungal growth after treatment while only 7% of the control samples (that did not receive any treatment) had no fungal growth.

### EXAMPLE 6

An additional protocol may be used in place of, or with, the protocol described in Example 3 above. The additional protocol is shown as flow charts in FIGS. 36-38.

Referring to FIG. 36, the system may be calibrated by first installing a patient at step 3610. The patient presses a button to start the calibration protocol at step 3615. An initial power of the electromagnetic energy is set at step 3620. A first power value PTX is selected at step 3620 by the operator. In the next several steps, the energy level may be optimized. If T_{TOE} was greater than T_{CAL}_{_{—}}_{START} at step 3622, then a waiting period at step 3655 occurred. If T_{TOE} was not greater than T_{CAL}_{_{—}}_{START}, then the temperature T₀ was set at step 3625 to the toe temperature Tₜₒₑ and a five second delay occurred. The temperature T₅ was set at step 3630 to the toe temperature Tₜₒₑ and a ten second delay occurs. The temperature T₁₅ is set at step 3635 to the toe temperature Tₜₒₑ. Using the temperature values at T₁₅ and T₅, a dT/dt_{cal} value was obtained by subtracting the T₅ value from the T₁₅ value and dividing by the time (10 seconds) at step 3635. This value represents the slope of the temperature with respect to time. A suitable operating range for dT/dt_{cal} is about 0.35 to about 0.45° C./second. The dT/dt_{cal} value also reflects how well the nail and the applicator are coupled and how much energy is being supplied. If the dT/dt_{cal} value is less than or equal to a dT/dtₘₐₓ value at step 3640 and is greater than or equal to a dT/dtₘᵢₙ value at step 3645, then the system is ready to initiate treatment at step 3650. If, however, the dT/dt_{cal} value is greater than dT/dtₘₐₓ or the dT/dt_{cal} is less than dT/dtₘᵢₙ, and less than five calibration tries at step 3670 have been attempted, a new power setting is calculated at step 3665 and after the toe cools at step 3655, the calibration process is repeated beginning at step 3625. If five calibration tries have been attempted, then a calibrate retry condition may be generated at step 3675 so that user input may be prompted at step 3685. If the power exceeds a maximum power conditions at step 3660, user input may also be prompted at step 3185 and treatment may be interrupted at step 3690.

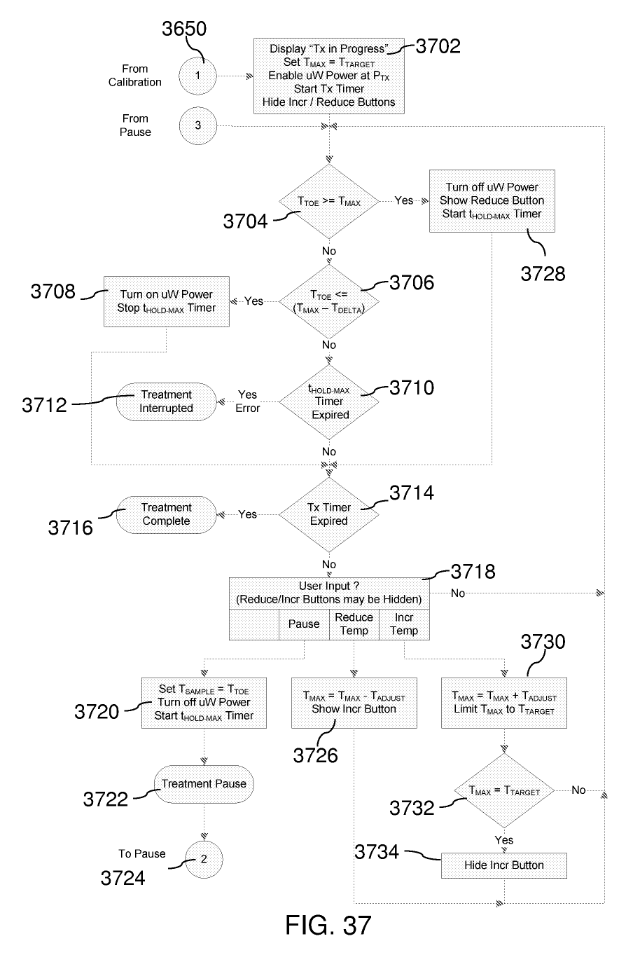

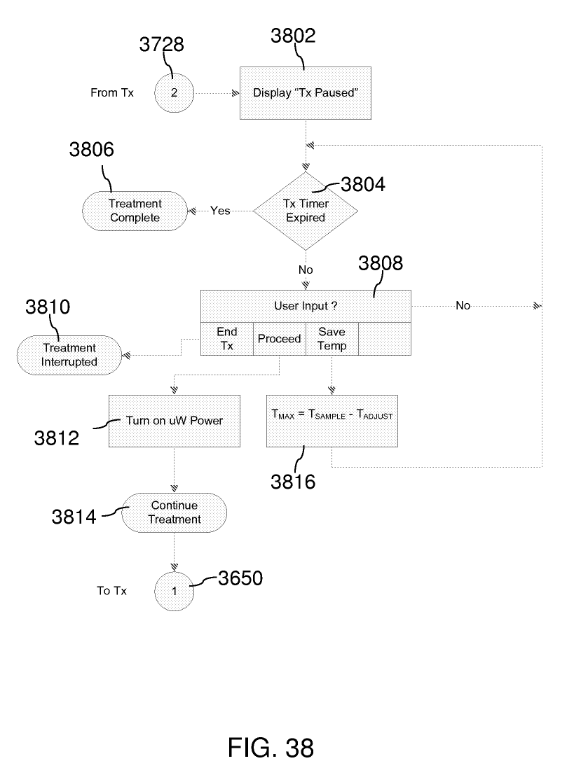

Once the system is calibrated treatment may begin as shown in FIG. 37. The treatment protocol shown in FIG. 37 is based on a predetermined treatment time, whereas the treatment protocol shown in FIG. 32 is based on a predetermined time that the energy is actively delivered to the target. Referring to FIG. 37, from the calibration protocol 3650, the maximum treatment temperature T_{MAX} is set to the target temperature T_{target} at step 3702. The energy power is also enabled at step 3702, and a timer Tₓ is started at step 3702 as well. The buttons on the user interface may also be hidden at step 3702 to prevent the user from changing the treatment parameters. If the toe temperature T_{TOE} does not exceed or is not equal to the maximum temperature T_{MAX} at step 3704, then the system proceeds to step 3706. If the toe temperature T_{TOE} does exceed or is equal to the maximum temperature T_{MAX} at step 3704, then the power is turned off at step 3728. The user controls may also be displayed at step 3728 and a timer t_{HOLD-MAX} is started. At step 3706, if the toe temperature T_{TOE} is not less than or equal to the maximum temperature minus a change in temperature T_{DELTA}. T_{DELTA} is selected to maintain an average temperature +/− 1° C. then the system proceeds to step 3710. If at step 3706 the toe temperature T_{TOE} is less than or equal to the maximum temperature minus a change in temperature T_{DELTA}, then the power is turned on at step 3708, a t_{HOLD-MAX} timer is stopped and treatment begins and continues until the timer Tₓ expires at step 3714 and treatment is considered complete at step 3716. If at step 3710, the timer t_{HOLD-MAX} has expired, then the system proceeds to step 3712 and treatment is interrupted and an error may be generated. If at step 3710, the timer t_{HOLD-MAX} has not expired, then the system proceeds to step 3714. If the Tₓ timer has not expired, then the system proceeds to step 3718 where user input may be required or the system may return at step 3704 for treatment. If user input is required, the user may select to stop or pause the treatment, and the power is turned off at step 3720, a timer t_{HOLD-MAX} is started, a sample temperature Tₛₐₘₚₗₑ is set to the toe temperature Tₜₒₑ, and treatment is paused at step 3722. The system may then proceed to step 3724 as shown in FIG. 38. If a user chooses to reduce the temperature at step 3718, then the maximum temperature T_{MAX} may be adjusted by T_{ADJUST} at step 3762 and the system may return to step 3704 for treatment. If the user desires to increase the temperature at step 3718, then the system may increase the maximum temperature T_{MAX} by T_{ADJUST}, and if T_{MAX} is not equal to the target temperature T_{TARGET} at step 3732, the system may return to step 3704 for treatment. If the T_{MAX} is equal to the target temperature T_{TARGET}, the user input buttons may be hidden at step 3734, and the system may return to step 3704 for treatment.

Referring to FIG. 38, if treatment is paused at step 3724, then the system may display a message at step 3802. If the timer Tₓ has expired at step 3804, then treatment is complete at step 3806. If the timer Tₓ has not expired at step 3806, then the system proceeds to step 3808 for user input. If the user selects to end treatment, then the system proceeds to step 3810 and treatment is interrupted. If the user selects to continue treatment, then the system proceeds to step 3812 where the power is turned on. Treatment is continued at step 3814 and the system returns to step 3650 and proceeds through the protocol described above in reference to FIG. 37.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

What is claimed is:

1. A system constructed and arranged to treat a mammalian tissue infected with an organism, the system comprising:
an electromagnetic energy source;
an applicator operatively coupled to the electromagnetic energy source and configured to deliver electromagnetic energy to the mammalian tissue; and
a controller operatively coupled to the electromagnetic energy source and configured to determine a treatment dose of the mammalian tissue and to provide for delivery of the determined treatment dose of the electromagnetic energy to the mammalian tissue.

2. The system of claim 1, further comprising a temperature sensor operatively coupled to the controller and configured to detect a treatment temperature.

3. The system of claim 1, in which the applicator comprises an adaptor constructed and arranged to be placed in proximity to the tissue to deliver the electromagnetic energy.

4. The system of claim 1, in which the adaptor is constructed and arranged to conform to a digit surface.

5. The system of claim 4, in which the digit surface is a nail.

6. The system of claim 1, in which the applicator comprises a tissue interface configured to receive a bolus.

7. The system of claim 6, in which the tissue interface is configured to provide impedance matching of the mammalian tissue and the applicator.

8. The system of claim 1, in which the applicator comprises a flexible substrate configured for a single use.

9. The system of claim 1, in which the controller is configured to provide pulses of the determined treatment dose.

10. The system of claim 1, in which the controller is configured to provide the determined treatment dose to provide continuous heating of the tissue until the mammalian tissue reaches a treatment temperature.

11. The system of claim 10, in which the controller is configured to halt delivery of the determined treatment dose once the mammalian tissue reaches the treatment temperature.

12. The system of claim 11, in which the controller is configured to continue delivery of the determined treatment dose once the mammalian tissue drops below the treatment temperature.

13. The system of claim 1, in which the controller is configured to deliver the determined treatment dose for a selected time.

14. The system of claim 3, in which the adaptor is constructed and arranged to smooth the distribution of energy.

15. The system of claim 5, in which the adaptor is constructed and arranged to treat at least two nails simultaneously.

16. A method of treating a tissue of a mammal infected with an organism, the method comprising:
a first step comprising determining a treatment dose of electromagnetic energy that a mammal can tolerate; and
a second step comprising exposing the tissue to the determined treatment dose for a treatment time.

17. The method of claim 16, further comprising a third step comprising halting exposure of the tissue to the determined treatment dose once the tissue reaches a first temperature.

18. The method of claim 17, further comprising a fourth step comprising continuing exposure of the tissue to the determined treatment dose once tissue temperature drops below the first temperature.

19. The method of claim 18, in which the steps of halting and continuing are repeated for the treatment time.

20. The method of claim 16, further comprising obtaining a culture of an organism infecting the tissue to assess efficacy of treatment.

21. The method of claim 19, further comprising assessing efficacy of treatment in less than one month or two weeks following the treatment.

22. The method of claim 16, further comprising exposing the tissue to one or more power levels of electromagnetic energy to determine the rate of heating to the first temperature.

23. The method of claim 19, further comprising removing an onycholytic portion of a nail before treatment.

24. The method of claim 19, further comprising placing a biocompatible material over treated tissue to block access of infectious agents after treatment.

25. The method of claim 24, in which the biocompatible material is toxic to infectious agents.

26. The method of claim 19, further comprising delivering a drug to the infected tissue with the electromagnetic energy provided to the tissue by iontophoresis.

27. The method of claim 19, further comprising delivering a drug to the infected tissue with the electromagnetic energy provided to the tissue by dielectrophoresis.

28. The method of claim 16, further comprising exposing the tissue to the determined treatment dose for the treatment time from about five minutes to about thirty minutes.

29. The method of claim 18, further comprising increasing the first temperature during treatment based on a new tolerance level of the mammal.

30. The method claim 18, further comprising increasing temperature of the first temperature by inducing reactive hyperemia in the tissue.

31. The method of claim 18, further comprising increasing temperature of the first temperature by exposing the tissue to a coolant blown or sprayed on or encompassing the tissue.

32. The method of claim 18, further comprising increasing temperature of the first temperature by exposing the tissue to a vibrating motion.

33. The method of claim 18, in which the determined treatment dose is effective to increase a nail growth rate.

34. A kit for treating an infected tissue, the kit comprising:
an adaptor constructed and arranged to be coupled to an electromagnetic energy source and to deliver electromagnetic energy to an infected tissue;
a bolus configured to focus the electromagnetic energy to the infected tissue; and
instructions for using the adaptor and the bolus to treat the infected tissue.

35. The kit of claim 34, in which the adaptor further comprises a tissue interface configured to receive the bolus.

36. The kit of claim 34, in which the adaptor is constructed and arranged to treat a nail.

37. The kit of claim 34, in which the adaptor is constructed and arranged to treat a hoof.

38. The kit of claim 34, in which the bolus is configured to provide impedance matching of the infected tissue and the adaptor.

39. A system constructed and arranged to treat a digit surface tissue infected with an organism, the system comprising:
an electromagnetic energy source;
an applicator operatively coupled to the electromagnetic energy source and configured to deliver electromagnetic energy to the digit surface, the applicator comprising a tissue interface configured to receive a bolus, and an adaptor coupled to the applicator constructed and arranged to conform to the digit surface; and
a controller operatively coupled to the electromagnetic energy source and configured to provide for delivery of a determined treatment dose of the electromagnetic energy to the digit surface.

40. The system of claim 39, in which the adaptor is constructed and arranged to conform to a nail.

41. The system of claim 40, in which the adaptor is constructed and arranged to conform to a hoof.

42. The system of claim 39, in which the tissue interface is configured in combination with the bolus to smooth the distribution of the electromagnetic energy provided to the digit surface.

43. The system of claim 39, further comprising a temperature sensor operatively coupled to the digit surface and configured to detect a treatment temperature.

44. A system for treating a mammalian nail or hoof infected with an organism, the system comprising:
an applicator constructed and arranged to deliver electromagnetic energy to a nail or a hoof; and
a housing sized and arranged to receive a hand, a foot or a hoof of a mammal, the housing comprising
an electromagnetic energy source operatively coupled to the applicator, and
a controller operatively coupled to the electromagnetic energy
source, configured to determine a treatment dose of the nail or hoof, and configured to provide for delivery of the determined treatment dose of electromagnetic energy to the nail or the hoof.

45. The system of claim 44, in which the applicator comprises a plurality of adaptors to treat at least two adjacent digit surfaces on the hand, foot or hoof.

46. The system of claim 45, in which at least one adaptor of the plurality of adaptors comprises a tissue interface configured to receive a bolus.

47. The system of claim 46, in which the tissue interface in combination with the bolus is configured to provide impedance matching of the mammalian tissue and the applicator.
